(19) United States
(12) Patent Application Publication
(10) Pub. No.: US 2026/0263700 A1
(43) Pub. Date: Sep. 10, 2026

(54) INJECTION DEVICE WITH REDUCED AMOUNT OF PARTS

(71) Applicant: Ypsomed AG, Burgdorf (CH)

(72) Inventors: Oliver Glauser, Bern (CH); Dominik Zumstein, Lyss (CH); Simon Martin Bosshard, Hindelbank (CH); Christian Schrul, Oberburg (CH); Markus Tschirren, Burgdorf (CH); Peter Loser, Herrenschwanden (CH); Andres Mellenberger, Koppigen (CH)

(21) Appl. No.: 19/665,498

(22) Filed: May 1, 2026

Related U.S. Application Data
(63) Continuation of application No. PCT/EP2024/079008, filed on Oct. 15, 2024.

(30) Foreign Application Priority Data
Nov. 7, 2023 (EP) 23208238.8

(51) Int. Cl. A61M 5/315; A61M 5/32

(52) U.S. Cl. CPC A61M 5/31576; A61M 5/3204

(57) ABSTRACT

An injection device includes a pre-assembled drive unit connected to a pre-assembled syringe unit. The pre-assembled syringe unit includes a syringe holder and a needle cover and the pre-assembled drive unit includes a plunger rod, a spring member adapted bias the plunger rod in a dispensing direction, a supporting member, and a first and a second retaining element, where each retaining element is configured to be arranged in a holding position in which the first or second retaining element holds the biased plunger rod in place relative to the supporting member and in a release position in which the first or second retaining element does not hold the biased plunger rod in place.

### CROSS-REFERENCE TO RELATED APPLICATIONS

This application is a continuation of International Patent Application No. PCT/EP2024/079008, filed Oct. 15, 2024, entitled “INJECTION DEVICE WITH REDUCED AMOUNT OF PARTS,” which in turns claims priority to European Patent Application No. 23208238.8, filed Nov. 7, 2023, entitled “INJECTION DEVICE WITH REDUCED AMOUNT OF PARTS”, each of which is incorporated by reference herein, in the entirety and for all purposes.

### TECHNICAL FIELD

The present disclosure relates to drug delivery devices or medicament delivery devices with a pre-assembled drive unit connected to a pre-assembled syringe unit for use in injecting, delivering, administering, infusing or dispensing substances and/or liquids such as insulin, hormone preparations or vaccines.

### BACKGROUND

A variety of diseases exist that require regular treatment by subcutaneous administration of a medicament, and a number of drug delivery devices have been developed to support a patient in accurately and controllably delivering an amount of drug in a self-administration process. Delivery devices include injection devices that are removed from the injection site after each medication event or drug delivery process, as well as infusion devices with a cannula or needle that remains in the skin of the patient for a prolonged period of time.

By way of example, diabetes may be treated by self-administration of insulin or its derivatives with the help of multi-variable-dose insulin injection pens. An injection pen device generally has an elongate device body defining a longitudinal main device axis. An automatic injection device has a motor or a drive spring for biasing a plunger rod and shifting a piston in a container barrel, where the drive spring may have to be charged or strained manually prior to injection of a dose. A manually powered delivery drive requires a user to manually provide the energy to move the piston, for instance by applying a distal force component to the injection device.

The medicament dose to be injected may be manually selected by turning a dosage knob and observing the actual dialed dose from a dose window or display of the insulin pen. A dose is dispensed by inserting the needle into a suited portion of human skin and by moving the piston manually or by pressing a release button of an automatic injection device. Automatic injection devices may comprise an electronic dose dial mechanism to automatically set a dose.

Also known is the use of autoinjectors comprising prefilled syringes. Autoinjectors usually comprise a body for housing the syringe as well as an automatic drive mechanism to move the plunger of the syringe upon actuation of the autoinjector. The drive mechanism of the autoinjector typically includes a source of drive, such as a strong spring for moving a drive member, for example a rod, which acts on the plunger of the syringe. The prefilled syringe has a needle or cannula that is permanently attached to a first end of a syringe barrel or reservoir that is sealed at the opposing end by the plunger.

For safety and hygiene reasons it is desirable that the needle does not protrude from a housing of the autoinjector with the exception of the time when the needle is used for injection of a medicament. Therefore, either the autoinjector moves the needle out of the housing for the injection and back into the housing after injection or the autoinjector provides a needle cover or needle guard which may be moved to unsheathe the needle for an injection, and which may be moved back in a needle covering position after the injection.

The majority of autoinjectors are configured as single use devices which incorporate both the syringe and the drive mechanism in the same housing. Such devices are usually disposable for ease of use.

In order to reduce costs and complexity it is desirable to design injection devices with a small number of parts and components. From an ecological aspect it is desirable to reduce the waste and to reuse devices as long as possible. In order to account for both ecological responsibility and medical and safety standards autoinjectors are developed as two-part or semi-disposable or semi-reusable devices. Such autoinjectors comprise a part which includes the syringe or reservoir with the drug, and which can be separated and disposed of separately from the other part, for example, from a drive unit including a drive mechanism of the autoinjector.

EP 3930793 A1 discloses an autoinjector with a housing which can be separated after use of the autoinjector. A first housing part houses the syringe, a plunger rod and parts of the drive mechanism and a second housing part includes an electronic unit including a sensor, a PCB and a power supply. After use the second housing part with the electronics can be disposed of separately from the first housing part allowing for a correct waste separation.

EP 3019220 B1 discloses an autoinjector comprising a drive unit and a control unit which are assembled after a syringe has been inserted into the housing. When the autoinjector is pressed against an injection site, a needle shroud is moved proximally causing a plunger rod to rotate relative to the housing from an initial position to a release position. After the rotation the plunger rod is free to move axially in the dispensing direction under the force of a drive spring.

WO 2010/000559 A1 discloses an autoinjector comprising a tubular elongated housing. Inside a hollow plunger rod is arranged wherein a spiral spring is arranged within the plunger rod. A guide for the springe is arranged inside the spring. The autoinjector includes a rotatable manual knob at a proximal end. Rotation of the knob causes a rotation displacement of a container holder and this in turn causes mixing of the medicament.

GB2560558A discloses an autoinjector with a housing and a trigger button inserted into a rearward portion of the housing to close the rearward end of the hosing. The button has a cup-like profile with side walls. The housing includes a drive mechanism comprising a plunger rod which is arranged to engage a bund inside the syringe. The plunger rod is driven in the dispensing direction by a pair of concentric drive springs.

### SUMMARY

It is an objective of the disclosure to provide an autoinjector with a simple design which is easy to assemble, and which is optimized for waste separation after use.

This objective is achieved by an injection device or a method according to the claims and the disclosure.

The disclosure more particularly relates to a method of assembling an injection device, and to an injection device for dispensing a liquid drug from a prefilled syringe through an injection needle permanently attached to a syringe barrel. The injection device includes a pre-assembled drive unit connected to a pre-assembled syringe unit. The pre-assembled syringe unit includes:
a housing defining a longitudinal axis;
a syringe holder adapted to hold the syringe such that the syringe cannot move relative to the syringe holder or relative to any other part of the housing;
a needle cover movable along the longitudinal axis relative to the syringe holder between a needle covering position in which the needle cannot be accessed from outside and a retracted position in which the needle protrudes from the needle cover,
The pre-assembled drive unit includes:
a plunger rod;
a spring member adapted to bias the plunger rod in a dispensing direction and adapted to move the plunger rod in the dispensing direction during injection;
a supporting member adapted to support or guide the spring member or/and the plunger rod;
a first and a second retaining element, where each of the first and second retaining element is configured to be arranged in a holding position in which the first or second retaining element holds the biased plunger rod in place relative to the supporting member and in a release position in which the first or second retaining element does not hold the biased plunger rod in place.

Prior to assembly of the injection device, the pre-assembled drive unit and the pre-assembled syringe unit are separated from each other and the first retaining element is in its holding position. Therefore, the biased plunger rod is exclusively held in place by the first retaining element in the pre-assembled drive unit.

During connection of the pre-assembled syringe unit to the pre-assembled drive unit, a movement of the needle cover or of the housing (and syringe holder) relative to the supporting member causes the first retaining element to move out of its holding position into a release position, such as by a deflection or release movement of the first retaining element from the plunger rod.

When the injection device is completely or final assembled, the drive unit is connected to the syringe unit and the needle cover is operatively coupled (directly or indirectly via intermediate member) to the second retaining element, where:
the first retaining element is in its released position and thus no longer holds the plunger rod, and
the second retaining element is in its holding position such that the biased plunger rod is exclusively held in place by the second retaining element when the needle cover is in its covering position.

Prior to assembly of the injection device, the syringe unit and the drive unit are pre-assembled. That means the pre-assembled syringe unit and the pre-assembled drive unit can be stored, handled and transferred as separate units before a final assembly step. This facilitates the storage and manufacture of the injection device.

The syringe holder or/and the needle cover may include a holding member or a holding means for holding the syringe holder and the needle cover together, such that they cannot fall apart and cannot be separated. The holding member or holding means may be, for example, a snap-fit element, a clip or a thread. The same applies for the pre-assembled drive unit. That means the plunger rod, the spring member, the supporting member and the first and second retaining elements are held together by a holding member. The components of the syringe unit and the components of the drive unit, respectively, can thus be handled and stored together.

Furthermore, the pre-assembled drive unit and the pre-assembled syringe unit enable a simple and quick final assembly of the injection device.

The pre-assembled drive unit and the pre-assembled syringe unit may be connected in a temporary or provisional way, different from the permanent connection formed upon final assembly, and easily releasable. The temporary or provisional connection may be established at the pre-assembly site, and the provisionally connected drive unit and syringe unit may be transported to a different location for final assembly. This allows for space efficient and economic shipping or transportation and decreases the complexity of final assembly, as for the final assembly step, a worker or machine only has to release the provisional connection, insert the syringe and assemble the drive unit and the syringe unit to obtain a complete injection device, ready for distribution.

After use, the syringe unit and the drive unit may be disconnected by a skilled person and such as by a dedicated tool rather than by the user. The tool-wise separation of the syringe unit from the drive unit provides, for example, a separation of components of different materials and thus a correct waste separation after use.

Moreover, the pre-assembled syringe unit and the pre-assembled drive unit according to the disclosure enable the plunger rod to be biased and held in place, e.g., held in a biased state, prior to assembly, during assembly and in the completely assembled injection device. Namely, the first retaining element holds the plunger rod in place prior to assembly. During assembly, the holding function is transferred from the first holding element to the second holding element such that in a final state the plunger rod is exclusively held in place by the second retaining element and is no longer held by the first retaining element.

After assembly, the biased plunger rod is configured to be held in place depending on a needle cover position (retraced or covering position) as the second retaining element is directly or indirectly coupled to the needle cover when the syringe unit is connected to the drive unit.

During assembly, at least a part of the drive unit is inserted into the syringe unit. A relative movement between the needle cover and the supporting member transfers the function of holding the plunger rod in place from the first to the second retaining element. That means a handing over of the held and biased plunger rod is quasi automatically conducted as no user action or additional assembly step is required to bring the first retaining element into its release position and subsequently to bring the second retaining element into its holding position. This further facilitates the assembly process of the injection device.

The relative movement during assembly of the injection device moves the first retaining element from its holding position into its released position. This may be, for example, by a deflection or by a disengagement or release movement of the retaining element. By way of example, a cam, protrusion or release member on a counterpart may contact or engage the first retaining element during the relative movement to bring the first retaining element in its released position.

During the period when the first retaining element is released and brought out of its holding position or subsequently or straight after this release, the second retaining element is brought into its holding position or kept in its holding position. That means the second retaining element may be in its holding position or it may be actively brought into its holding position such that it can hold the biased plunger rod in place. During this transition phase, the spring member may expand a very short distance, for example 1 mm or less.

Additionally, the second retaining element is directly or indirectly coupled to the needle cover, for example, by a coupling sleeve or by a coupling element such as a coupling cam, hook, protrusion or snap fit element during connection of the syringe unit with the drive unit.

The first and second retaining elements are adapted to hold the biased plunger rod in place such that it cannot move in the dispensing direction. The first and second retaining elements may be implemented, for example, in form of a clamp, holding or retaining arm, protrusion or form-fit element.

The supporting member is adapted to support and guide the plunger rod along the longitudinal axis and may guide the plunger rod such that it can move exclusively along the longitudinal axis and prevents a rotational or radial movement of the plunger rod. The supporting member may include, for example, a rib, a groove. Instead, or additionally, the supporting member may support or guide the spring member such that a reliable compression and relaxation of the spring member is enabled.

When the pre-assembled drive unit is connected to the pre-assembled syringe unit, an injection device is formed. That means the syringe unit or the drive unit alone are not considered to be an injection device. The injection device is produced if the pre-assembled syringe unit is connected to the pre-assembled drive unit.

The injection device may be a disposable automatic injection device such as an autoinjector. Autoinjectors usually include a syringe and an automatic drive mechanism to move a piston inside the syringe upon actuation of the autoinjector. The syringe may be a prefilled syringe that has a needle or cannula permanently attached to a distal end of a syringe barrel or reservoir and that is sealed at the opposing end by a piston.

The spring member provides a spring force. By way of example, the spring member may be a metallic solenoid or spiral spring, a flat spring or leaf spring, or an elastomer element, or an elastically deformable plastic member adapted to provide a spring force in a biased state.

The syringe holder is adapted to hold the syringe with the liquid drug in place. That means the syringe holds the syringe relative to a housing and relative to the drive unit in a fixated manner and in particular prevents any axial movement of the syringe relative to the housing and drive unit during dose dispensing.

In the present context, the terms “substance”, “drug”, “medicament” and “medication” are to be understood to include any flowable medical formulation suitable for controlled administration through a means such as, for example, a cannula or a hollow needle, and includes a liquid, a solution, a gel or a fine suspension containing one or more medical active ingredients. A medicament can be a composition comprising a single active ingredient or a pre-mixed or co-formulated composition with more than one active ingredient present in a single container. Medication includes drugs such as peptides (e.g., insulin, insulin-containing drugs, GLP-1 containing drugs or derived or analogous preparations), proteins and hormones, active ingredients derived from, or harvested by, biological sources, active ingredients based on hormones or genes, nutritional formulations, enzymes and other substances in both solid (suspended) or liquid form but also polysaccharides, vaccines, DNA, RNA, oligonucleotides, antibodies or parts of antibodies but also appropriate basic, auxiliary and carrier substances

The term “distal” is meant to refer to the direction or the end of the injection device carrying an injection needle or an injection cannula, whereas the term “proximal” is meant to refer to the opposite direction or end pointing away from the needle or cannula.

The term “injection system” or “injector” refers to a device that is removed from the injection site after each medication event or drug delivery process, whereas the term “infusion system” refers to a device with a cannula or needle that remains in the skin of the patient for a prolonged period of time, for example, several hours.

In embodiments, the method of assembling includes the steps of inserting a distal portion of the pre-assembled drive unit into a proximal connecting portion of the housing of the pre-assembled syringe unit, and connecting the drive unit supporting member to the syringe unit housing by the connecting element to establish a snap-fit connection between the supporting member and the housing. The latter may only be released by dedicated equipment, for reuse or recycling purposes. In the assembled state of the injection device, the second retaining element is in its holding position when the needle cover is in the covering position, and the second retaining element is in its release position when the needle cover is in the retracted position. The second retaining element is thus coupled to the needle cover, or a position of the second retaining element is depending on the needle cover position.

That means the biased plunger rod is held in place when needle is covered and thus access to the needle is not enabled. When the user moves the needle cover from the covering position into the retracted position (for example by a push on skin activation), the plunger rod may be released, and the spring member can release, thereby moving the plunger rod in the dispensing direction to dispense the drug from the syringe.

The supporting member may further include:
A connecting element adapted to connect the pre-assembled drive unit to the pre-assembled syringe unit;
A guiding element adapted to guide the plunger rod along the longitudinal during dispensing. For instance, the guiding element may be adapted to guide the plunger rod such that the plunger rod cannot rotate relative to the supporting member.
A support surface for supporting the spring member at a proximal end of the spring member.

The supporting member as a single part thus fulfils a plurality of functions, namely a connecting, a guiding as well as a supporting function. This provides for a simple design and facilitates the assembly process as the number of components is reduced. Furthermore, the first and second retaining elements may be connected to or integrally formed in the supporting member. That again combines different functions in one single supporting member.

The connecting element to connect the drive unit to the syringe unit may be a deflectable snap-fit member or a recess adapted to accommodate a snap-fit member. That means the syringe unit provides the other of the snap-fit member or the recess. The connection by means of the snap-fit member and the recess is thus a form-fit connection. That enables a quick and reliable connection between the syringe unit and the drive unit. The snap-fit connection further enables a disconnection of the syringe unit from the drive unit after use, such as by a dedicated tool. The syringe unit and the drive unit may thus be separated in order to be disposed of separately.

The guiding element may be, for example, a rib, a groove, a rail, a dovetail guide or a form-fit engagement. The guiding element ensures a reliable movement of the plunger rod in the dispensing direction during injection. The support surface provides a proximal end support for the spring member.

The supporting member may include a sleeve-shaped portion including a circumferential wall defining a bore, where the biased plunger rod is at least partly arranged inside the bore. This enables a space-saving design and a facilitates the assembly as the spring member and the plunger rod may be accommodated at least partially inside the bore. If the supporting member includes a guiding element for the plunger rod the guiding element may be positioned within the bore.

Furthermore, in embodiments, the first and in some cases the second retaining element are arranged in the wall and may be integrally formed in the supporting member wall. Thus, the manufacturing of the supporting member (e.g., by injection moulding) is facilitated as the body of the supporting member and the retaining elements can be produced on one single production step.

In examples, the first retaining element is an arm extending along the longitudinal axis with a retaining portion on a free end of the arm. The retaining arm may be deflectable in a radial direction to release the plunger rod from its biased position such that the plunger rod can move in the dispensing direction. That means the first retaining arm is configured to be moved between its holding position and its release position by a radial deflection. The retaining portion of the arm may be a knob, a cam or a hook adapted to engage a recess or counter element in the plunge rod.

In embodiments, the second retaining element may also be an arm extending along the longitudinal axis with a retaining portion on a free end. The first and second retaining elements formed as arms provide a simple and reliable holding mechanism to hold the biased plunger rod in place.

In certain embodiments, the plunger rod is hollow and defines a cavity and the spring member is arranged inside the cavity. For instance, the supporting member further includes a guiding pin extending along the longitudinal axis inside the spring member and inside the cavity of the hollow plunger rod to guide the spring member. That provides a simple and space-saving design. Alternatively, the plunger rod may be rod-shaped and the spring member, in particular a spiral spring is arranged outside and may envelop the rod-shaped plunger rod.

The pre-assembled syringe unit may further include a sleeve-shaped housing, and the needle cover may be coaxially arranged inside the housing with the syringe holder arranged inside the needle cover. Thus, the housing is the outermost member of the pre-assembled syringe unit. Accordingly, when the injection device is assembled, the housing is the outermost member of the injection device.

A cross-section of the housing may be circular but may have alternatively an elliptic, polygonal or rectangular shape. The housing and the syringe holder may be coaxially arranged. Both parts may be directly connected to each other, for example, by a form-fit, snap-fit or a threaded connection preventing any relative movement between the syringe holder and the housing. As the housing is directly connected to the syringe holder there is no need for an intermediate member and thus the syringe unit is configured to be assembled with a reduced number of parts.

The housing and/or the syringe holder may include a guiding member to guide a movement of the needle cover along the longitudinal axis. However, the needle cover is held such that it cannot fall off and cannot be removed from the assembled housing and syringe holder. The syringe holder and/or the hosing may include two stop elements restricting a movement of the needle cover in both directions to prevent a separation of the latter.

In embodiments, the housing includes a release surface adapted to move the first retaining element out of its holding position and into its release position by the relative movement during connection of the pre-assembled syringe unit with the pre-assembled drive unit. The first retaining element may be deflected, rotated, screwed or linearly moved out of its holding position by the release surface. The release surface may be, for example, a ramp or a sloped surface.

The biased plunger rod is therefore released from the first retaining element. The plunger rod is subsequently held in place by the second retaining element of the supporting member. The second retaining element may be held in place and held in its holding position by a locking surface of the needle cover.

That means during assembly of the pre-assembled syringe unit to the pre-assembled drive unit, the first retaining element is released and the biased plunger rod is released. The plunger rod is then held in place by the second retaining element. The second retaining element is prevented from being moved out of its holding position and thus locked in the holding position by the locking surface of the needle cover.

The drive unit may include a second spring member adapted to bias the needle cover into the covering position. The second spring member may be held in a biased state by a holding element of the supporting member and arranged on an outside of the supporting member.

The biased second spring may act directly or indirectly on the needle cover to ensure that the needle cover is kept in its covering position before an injection and unintentional retraction of the needle cover may be prevented. To uncover the needle, the needle cover is moved from the covering position into the retracted position against a force of the second spring member.

The holding element may be, for example, a flexible arm, snap element or rim portion adapted to hold the second spring member in place in a biased state. The second spring member may be released from the holding element upon attachment of the drive unit to the syringe unit such that the second spring member can engage the needle cover.

In embodiments, a movement of the needle cover, in particular an axial movement of the needle cover, or more particularly of the locking surface relative to the second retaining element enables the retaining element to move out of its holding position.

The movement of the needle cover may be caused by a needle cover retraction movement, e.g., when the user presses a distal end of the injection device against the injection site, thereby moving the needle cover from the covering position towards the retracted position (also referred to push-on-skin activation). The needle cover moves along the longitudinal axis and the second retaining element is deflectable in a radial direction or a direction angled to the longitudinal direction.

The housing includes a connecting portion located proximally to the syringe holder, which connecting portion extends along the longitudinal axis at least a half, such as a full length of the axial length of the syringe holder. The connecting portion is adapted to accommodate and to be connected to the drive unit to form the injection device.

As the connecting portion extends along the longitudinal axis, the pre-assembled drive unit is configured to be at least partially inserted into housing connecting portion. For instance, the supporting member is enveloped almost along its full axial length. That means the connecting portion accommodates and envelops the supporting member and thus the drive unit when the drive unit is connected to the syringe unit. The drive unit is thus protected from environmental impacts without requiring an additional housing. Moreover, the assembly is facilitated as no additional outer housing is required.

The connecting portion includes a connecting element such as, for example, a snap arm, protrusion or recess to form a form-fit connection with the connecting element of a supporting member of the pre-assembled drive unit.

The drive unit may further include a lock sleeve movable relative to the supporting member and adapted to lock, in the assembled state, the second retaining element and in some cases additionally the first retaining element in its holding position such that the second retaining element and also the first retaining element cannot be brought into its release position when the injection device is assembled.

The lock sleeve thus ensures that the second retaining element (and in some cases also the first retaining element) remain in the holding position and thus the biased plunger rod is not unintentionally released. The lock sleeve may be arranged on a sleeve-shaped portion of the supporting member and is movable along the longitudinal axis to lock or unlock the first and second retaining elements.

During connection of the syringe unit with the drive unit, the lock sleeve may be moved in along the longitudinal axis by a member of the syringe unit thereby releasing the lock and moving the first retaining element out of its holding position.

The lock sleeve may be operatively coupled to the needle cover such that a movement of the needle cover is transferred to the lock sleeve and vice versa.

Before assembly, the lock sleeve is in a first or initial locking position and the needle cover spring is arranged between (a distal flange of) the lock sleeve and the supporting member.

During connection of the pre-assembled syringe unit with the pre-assembled drive unit, the lock sleeve is moved into an unlocking position in which the first retaining element is enabled to move from its holding position into its release position.

The lock sleeve includes a deflectable locking arm or a recess to engage a deflectable arm and the needle cover includes the other of the arm or the recess. The arm may be configured to deflect during insertion of the supporting member into a connecting portion of a housing of the syringe unit such that the arm engages the recess, thereby axially coupling the needle cover of the pre-assembled syringe unit to the lock sleeve of the pre-assembled drive unit.

In embodiments, when the needle cover is moved in its retracted position, the lock sleeve is moved in a proximal end position where a return movement of the lock sleeve back in the distal direction is prevented by the first or second retaining element. In the proximal end position, the lock sleeve may be adapted to lock the needle cover in the covering position by a snap-fit connection arranged in a distal portion of the lock sleeve and in a proximal portion of the needle cover. This prevents a second retraction of the needle cover and thus provides for a reliable needle protection.

In embodiments, the supporting member includes an elastic element on its distal end adapted to bias the syringe inside the syringe holder in an assembled state of the injection device. The elastic element may be integrally formed in the supporting member such that the elastic element and the supporting member from a monolithic part.

That provides a simple and compact design. Furthermore, the supporting member and the elastic element is configured to be produced in only one production step, in particular by injection moulding. The elastic element may be formed by windings of spiral-shaped element.

The supporting member, the first and second retaining element, the plunger rod and, if any, the lock sleeve of the pre-assembled drive unit may be made of the same material, such as polyoxymethylene (POM). This enables disposal of the supporting member after use together with the plunger rod in the same manner or in the same waste stream. Polyoxymethylene may be selected in view of its friction, costs and availability.

Moreover, the syringe holder, the needle cover and, if any, the housing, of the pre-assembled syringe unit may be made of the same material. These components may be made of polypropylene (PP) in view of its costs and availability.

The disclosure further relates to a method of assembling an injection device for dispensing a liquid drug from a syringe through an injection needle, from a pre-assembled syringe unit with
a housing defining a longitudinal axis,
a needle cover movable along the longitudinal axis relative to the housing between an expanded, needle-covering position and a retracted, needle-exposing position, and from a pre-assembled drive unit with:
a plunger rod,
a spring member adapted to bias the plunger rod,
a supporting member adapted to support the spring member,
a first, pre-assembly retaining element retaining the plunger rod relative to the supporting member against the bias of the spring member, and
a second, post assembly retaining element or mechanism adapted to retain, in a locked state,
the plunger rod relative to the supporting member against the bias of the spring member, the method including the steps of:
inserting the drive unit into the syringe unit,
biasing the needle cover into the expanded position by means of a needle cover spring of the injection device,
locking the second retaining element by operatively coupling the second retaining element to the needle cover sleeve in the expanded position,
releasing the first retaining element such that the biased plunger rod is exclusively retained against the bias of the spring member by the locked second retaining element.

The second retaining element or mechanism may include a latching component that may undergo a release movement in a plane perpendicular to the longitudinal axis, in a radial or circumferential direction. Non-axial guide or camming surfaces of the piston rod and/or the latching component deflect the axial bias of the injection spring member on the piston rod and urge the latching component into the release movement. Locking the second retaining element against or preventing the second retaining element from unintended release of the plunger rod includes an axially extending, part-circumferential or radial locking surface of the needle cover, or of the lock sleeve coupled to the needle cover, locking a cam of the latching component against a perpendicular release movement. The second retaining element or mechanism may include non-axial guide or camming surfaces of the supporting member and/or the plunger rod, guiding the plunger rod into a rotative release movement under the bias of the injection spring member. Locking the second retaining mechanism includes an axially extending guide rail of the needle cover or the plunger rod locking an axially nonextended cam of the plunger rod or needle cover, respectively, against rotation. The second release element may not release the plunger rod unless the needle cover as a trigger element is retracted or activated.

The present disclosure further relates to a pre-assembled syringe unit connectable to a drive unit as described herein to assemble or build an injection device for dispensing a liquid drug from a reservoir through an injection needle. The syringe unit includes:
a housing defining a longitudinal axis;
a syringe holder adapted to hold a syringe non-movable along the longitudinal axis relative to the housing; and
a needle cover movable along the longitudinal axis relative to the syringe holder between a needle covering position and a retracted position.

The syringe holder is directly connected to the housing, for example by a form-fit or snap-fit connection, such that the syringe holder cannot move relative to the housing. The needle cover is supported or held between the syringe holder and the housing such that the needle cover can move along the longitudinal axis between the covering position and the retracted position and cannot fall off or cannot separated from the syringe holder or from the housing.

As previously described in detail, the housing of the pre-assembled syringe unit may include, on a proximal side of the syringe holder, a connecting portion, which extends along the longitudinal axis at least a half, such as a full length of the axial length of the syringe holder and the connecting portion is adapted to accommodate the supporting member and is configured to be connected to the drive unit.

As the needle cover is held by the syringe holder and/or the housing, the needle cover cannot drop off and the pre-assembled syringe unit is configured to be stored and handled as one single unit or component. This facilitates handling and assembly.

Due to the pre-assembled syringe unit, the injection device is configured to be assembled by inserting a syringe into the syringe holder and by connecting the pre-assembled syringe unit with the pre-assembled drive unit. Hence, the two pre-assembled units enable a simple and quick final assembly of the injection device.

The disclosure further relates to a pre-assembled drive unit for an injection device for dispensing a liquid drug from a syringe through an injection needle, where the drive unit is connectable to a pre-assembled syringe unit and where the drive unit includes:
a plunger rod movable along a longitudinal axis;
a spring member for biasing the plunger rod in a dispensing direction, where the drive unit further includes a supporting member including a connecting element to connect the drive unit to the pre-assembled syringe unit;
a guiding element adapted to guide the plunger rod along the longitudinal axis;
a first movable retaining element for holding the biased plunger rod in place in a shipping state and prior to a connection of the drive unit with the syringe unit;
a support surface adapted to support the spring member at a proximal end of the spring member,
   and where the supporting member further includes a sleeve-shaped portion including a circumferential wall defining a bore where the biased plunger rod is at least partially arranged inside the bore. The first retaining element may be arranged in the wall.

In a further embodiment of the disclosure, the second retaining element is a deflectable arm including a first and a second cam oppositely arranged on a free end of the arm, where the first cam is adapted to engage the plunger rod and the second cam is adapted to engage a locking surface of the needle cover in an assembled state of the injection device.

In this embodiment the needle cover may be sleeve-shaped and includes a guiding track (next to the locking surface) adapted to guide the second cam, where the guiding track includes an enabling portion enabling a movement of the needle cover sleeve relative to the supporting member as the second cam can travel within the guiding track. Additionally, the guiding track includes a lock portion adapted to prevent movement of the needle cover sleeve relative to the supporting member as a movement of the second cam is blocked (e.g., by a stop surface) in the lock portion. The guiding track may be U-shaped.

The deflectable arm may be adapted to deflect radially in a first plane through a center longitudinal axis of the injection device and the arm is additionally adapted to deflect in a second plane offset the center longitudinal axis and perpendicular to the first plane.

Furthermore, the supporting member may include a snap element adapted to hold a needle cover spring in a biased state between the snap element and a proximal end flange of the supporting member.

In a further embodiment the first retaining element is a cam or a recess adapted to accommodate the cam, where the cam or recess is arranged on the plunger rod and the other of the cam and the recess is arranged on the supporting member.

The second retaining element may also be a cam or a recess adapted to accommodate the cam, where the cam or recess is arranged on the plunger rod and the other of the cam and recess is arranged on the needle cover.

The plunger rod may be rotatable relative to the supporting member and in a first rotational orientation of the plunger rod relative to the supporting member, the first retaining element is in its holding position (and the second retaining element may be in its release position) and in a second rotational orientation of the plunger rod, the first retaining element is in its release position (and the second retaining element may be in its holding position).

The plunger rod includes a sloped wall or an engagement element adapted to engage the sloped wall, and the needle cover or the housing includes the other of the sloped wall or engagement element, and when the plunger rod is moved in the dispensing direction, the engagement element slides along the sloped wall, thereby causing the plunger rod to rotate relative to the supporting member to move between the first and second rotational orientation.

In a further embodiment, the syringe unit includes a needle cover lock member having a recess and a stop wall. The lock member may be rotatable relative to the needle cover (and relative to the supporting member) and where the lock member is rotationally coupled to the plunger rod. In the first rotational orientation of the plunger rod, the recess of the lock member is rotationally aligned with a protrusion of the needle cover such that the needle cover is free to move between the covering position and the retracted position. In the second rotational orientation of the plunger rod, the lock member stop wall is rotationally aligned with the protrusion such that the needle cover can move in its covering position but is prevented from moving back into its retracted position.

In an alternative embodiment, the plunger rod is exclusively rotated during assembly of the injection device when the pre-assembled syringe unit is connected to the pre-assembled drive unit. In particular, the plunger rod is rotatable to release the first retaining element and to engage the second retaining element as previously described. In this alternative embodiment, the plunger rod is held rotationally fixed relative to the supporting member during the injection process. To release the plunger rod for the injection, the second retaining element may be moved out of its holding position (deflected, rotated, axially moved) such that the plunger rod is free to move axially in the dispensing direction without rotation.

The present disclosure further relates to a needle cover lock for an injection device for dispensing a liquid drug from a syringe through an injection needle, the needle cover lock including:
a supporting member defining a longitudinal axis;
a needle cover movable along the longitudinal axis relative to the supporting member between
a needle covering position and a retracted position in which the needle protrudes from the needle cover,
a lock member movable relative to the supporting member and being in contact or coupled (directly or indirectly) with the needle cover such that an axial movement of the needle cover is transferred to the lock member,
   where the lock member includes a stop element adapted to selectively engage a counter element on the supporting member,
   where in a first rotational orientation of the lock member relative to the supporting member, the stop element does not encounter the counter element such that the lock member is movable along the longitudinal axis,
   where in a second rotation orientation of the lock member, the stop element encounters the counter element thereby preventing a movement of the lock member and the needle cover towards the retracted position,
   and where a movement of the needle cover from the retracted position towards its covering position (and a distal movement of lock member) causes the lock member to rotate from its first rotational orientation into its second rotational orientation.

This arrangement provides a simple and reliable lock of the needle cover in the covering position and thus an unintentional access to the injection needle can be prevented.

The lock member may be rotated between the first and second rotational orientation by a force provided by a spring. The spring may be, for example, a mechanical spiral spring or an elastomer element initially mounted to the supporting member.

The lock member may be sleeve-shaped and includes on its inside a guide track or a guiding cam adapted to be guided in the guiding track and the supporting member includes the other of the guiding cam and the guide track. Such a cam control with a cam and a guiding track provides a reliable control of the movements of the lock member. That means the lock sleeve is configured to be axially guided to prevent rotation, or the guiding track can be configured such that the lock sleeve rotates at a specific axial position.

The guiding cam may be a deflectable arm. The stop member may be arranged inside the guide track and the counter element is arranged on the cam.

The lock member has a first linear guide to axially guide the lock member in the first rotational orientation and a second linear guide to axially guide the lock member in the second rotational orientation. The linear guide may be the guide track or a separate guide element. The lock member may additionally include an angled guiding section causing the rotation from the first rotational orientation into the second rotational orientation when the lock member is moved distally. The guiding cam may be adapted to engage the angled guiding section to initiate the rotation of the lock member.

The stop element may be a sawtooth with a sloped surface adapted to let pass the deflectable arm on a first side and a stop surface adapted to encounter the counter element on a second side. The sawtooth-shaped stop member provides a reliable locking of the needle cover in the covering position.

The supporting member may include an elastic element on its distal end adapted to bias a syringe inside a syringe holder. The elastic element may be integrally formed in the supporting member as a monolithic part.

In embodiments, the needle cover lock further includes a needle cover spring adapted to bias the needle cover in its covering position (in the distal direction). The supporting member may include a holding element to couple the lock member and the supporting member such that the biased needle cover spring is configured to be held in a biased state between the lock member and the supporting member prior to assembly of the injection device. That means the supporting member, the lock member, and the needle cover spring form a pre-assembled unit which is configured to be stored and handled as one unit. Additionally, the needle cover spring may be used to provide a force to rotate the lock member if released as described above.

The disclosure relates further to the injection device including the previously described needle cover lock. The injection device may include a pre-assembled syringe unit and a pre-assembled drive unit. The two units are configured to be assembled, stored and handled separate from each other. The two units may be connected to each other to form the injection device. The pre-assembled syringe may include:
a housing defining a longitudinal axis,
a syringe holder arranged inside the housing and
the needle cover,
   where the pre-assembled drive unit includes:
a plunger rod,
a spring member adapted bias the plunger rod in a dispensing direction,
the lock member and
the supporting member.

The housing may be sleeve-shaped, and the needle cover may be coaxially arranged inside the housing and the syringe holder is arranged inside the needle cover.

The housing may include a connecting portion as described above located proximally to the syringe holder, which connecting portion extends along the longitudinal axis at least a half, such as a full length of the axial length of the syringe holder. The connecting portion is adapted to accommodate and to be connected to the drive unit.

Further, the disclosure relates to an acoustic feedback mechanism for an injection device, the injection device may be an autoinjector with a pre-tensioned injection spring and including a plunger rod adapted for advancing a piston in an axial direction to dispense the entire amount of liquid drug from a container through an injection needle. The acoustic feedback mechanism includes signalling recesses axially arranged as an array on the plunger rod and an elastically mounted feedback element with a nose or head adapted to sequentially engage the recesses to generate a sequence of click sounds. The feedback mechanism may have the sole function of generating acoustic feedback and may not be involved in retaining or securing the plunger rod prior to injection. The acoustic feedback mechanism may include a plurality of individually mounted feedback elements adapted to alternatingly engage the recesses of a same array one at a time, in a non-concurrent manner, such that only a single click sound generated by a single feedback element is hearable at any given time.

In embodiments, at least a first and a second axial array of recesses are arranged on the piston rod on distinct sides thereof. Correspondingly, at least a first and a second feedback element may be provided and adapted to engage the recesses of the first and the second array, respectively. A third and even a fourth axial array may be provided and equidistantly distributed around a circumference of the piston rod, such as to form angles of 120 or 90 degrees with each other when seen in the axial direction.

The first and the second feedback element may be axially aligned and the recesses of the first array may be axially shifted with respect to the recesses of the second array. The magnitude of the shift or the axial distance between recesses of distinct arrays may be about half of the distance between the elements of a single array in the case of two arrays, or about 1/n of the distance between the elements of a single array in the case of n arrays. Alternatively, the elastically mounted feedback elements of distinct arrays are shifted with respect to each other, i.e., arranged at distinct axial positions, while the recesses of the arrays are axially aligned. In case of two arrays, the number of recesses per array may be less than eight, or less than seven.

Leading recesses may be arranged distally to the arrays to accommodate the feedback elements prior to injection, where the feedback elements are in a state with less or no bias on the elastically mounted arms when compared to the bias exerted on the mounting at any time during feedback generation. To that end, the leading recesses are larger than the signalling recesses of the array, to accommodate a larger fraction of the noses on the distally oriented elastic arms than the signaling recesses. Specifically, the leading recesses may be wider, longer, and/or deeper, depending on which part of the nose first abuts the wall of the piston rod surrounding a signaling recess. A leading recess may even be shaped as a cut-out or through-hole traversing the wall of the piston rod.

The feedback element and the recesses may be adapted or shaped to prevent proximal movement of the plunger rod. This configuration is only compatible with mounting the plunger rod from a rear or proximal end, in the distal direction with respect to the feedback element.

### BRIEF DESCRIPTION OF THE DRAWINGS

The subject matter of the disclosure will be explained in more detail in the following text with reference to exemplary embodiments, which are illustrated in the attached drawings, in which:

FIG. 1 depicts a perspective view of the components of an autoinjector according to a first embodiment of the present disclosure;

FIG. 2 depicts a perspective view of a pre-assembled syringe unit and a pre-assembled drive unit of the first embodiment;

FIGS. 3a and 3b depict sectional views of a cap of the autoinjector of FIGS. 1 and 2;

FIG. 4 depicts a sectional view of the autoinjector during assembly of the syringe unit and the drive unit;

FIG. 5a depicts a sectional view of the autoinjector in an initial state;

FIG. 5b depicts a sectional view in a plane 90° rotated compared to the view of FIG. 5a;

FIG. 6 depicts a sectional view of the autoinjector with a cover sleeve in the retracted position;

FIG. 7a depicts a sectional view of the autoinjector just after dispensing;

FIG. 7b depicts a sectional view of an alternative version of the first embodiment of the autoinjector with a lock sleeve with proximally extending locking arms;

FIG. 8 depicts a sectional view of the autoinjector after dispensing with the needle cover sleeve locked in the covering position, where the section plane is rotated by 45° relative to the previous sectional views;

FIG. 9 depicts a perspective view of the components of an autoinjector according to a second embodiment of the present disclosure;

FIG. 10 depicts a perspective view of the syringe unit and drive unit according to the second embodiment;

FIGS. 11 and 12 depict, respectively, the drive unit and the plunger rod of the second embodiment;

FIGS. 13 and 14 depict a sectional view of the autoinjector of the second embodiment in an initial state;

FIGS. 15 and 16 depict the autoinjector after dispensing, with FIG. 15 depicting a sectional view of the autoinjector and FIG. 16 depicting a perspective view of the autoinjector after dispensing;

FIG. 17 depicts a perspective view of the components of an autoinjector according to a third embodiment;

FIG. 18 depicts a perspective view of the syringe unit and drive unit of the third embodiment;

FIGS. 19 and 20, respectively, depict perspective views of the lock sleeve and the supporting member;

FIGS. 21 and 22 depict a sectional view of the autoinjector of FIG. 18 in an initial state;

FIG. 23 depicts a sectional view of the autoinjector after dispensing with the cover sleeve in the retracted position;

FIG. 24 depicts a sectional view of the autoinjector after dispensing with the cover sleeve locked in the covering position;

FIG. 25 depicts a version of the third embodiment with a plunger rod including saw teeth;

FIG. 26 depicts a further version of the third embodiment with click arms sliding over the injection spring windings;

FIG. 27 depicts a perspective view of the components of an autoinjector according to a fourth embodiment;

FIG. 28 depicts a perspective view of the syringe unit and drive unit of the fourth embodiment;

FIGS. 29 and 30, respectively depict perspective views of the plunger rod and the supporting member;

FIG. 31 depicts a sectional view of the autoinjector during assembly;

FIG. 32 depicts a sectional view of the autoinjector in an assembled state;

FIG. 33 depicts a perspective view of the autoinjector during assembly;

FIG. 34 depicts a sectional view of the autoinjector in an assembled state;

FIG. 35 depicts a sectional view of the autoinjector after dispensing with the cover sleeve in the retracted position;

FIG. 36 depicts a sectional view of the autoinjector after dispensing with the cover sleeve locked in the covering position;

FIG. 37 depicts a fourth version of the third embodiment;

FIG. 38 depicts a section view of the fourth version of the third embodiment during injection, and

FIG. 39 depicts a section view of the fourth version of the third embodiment before injection.

The reference symbols used in the drawings, and their primary meanings, are listed in summary form in the list of designations. In principle, identical parts are provided with the same reference symbols in the figures.

### DETAILED DESCRIPTION

In the following the term “distal” refers to the side where an injection needle is located. That is the left-hand side in the figures. The term “proximal” refers to the opposite side which is furthest away from the needle which is the right-hand side in the figures.

### First Embodiment

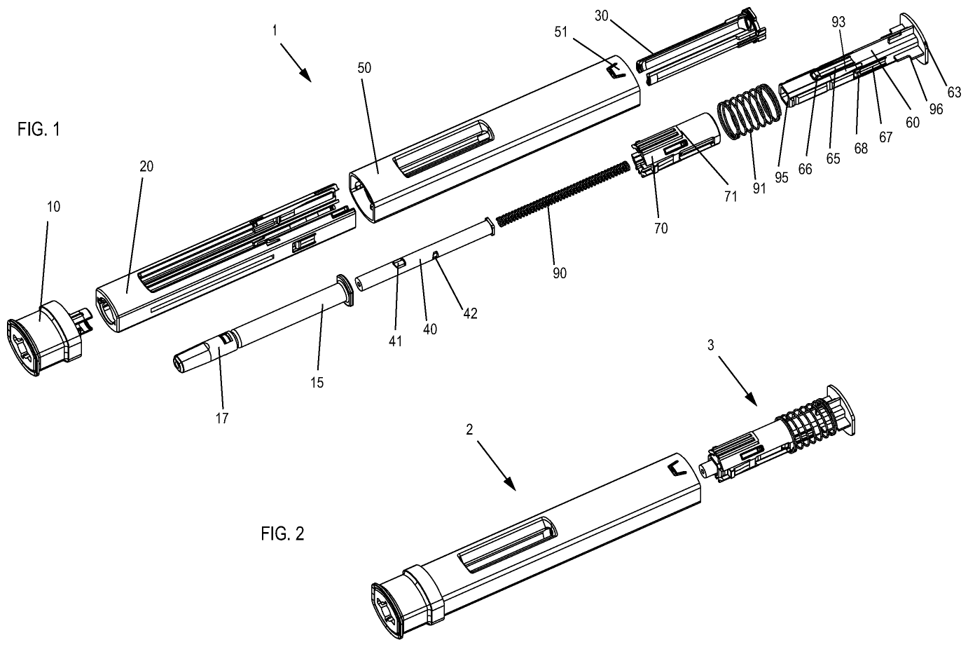

FIG. 1 depicts a perspective exploded view of an injection device according to a first embodiment of the present disclosure in the form of an autoinjector 1. The autoinjector 1 includes a pre-assembled syringe unit 2 and a pre-assembled drive unit 3 (FIG. 2). During assembly a prefilled syringe 15 is inserted into a syringe holder 30 and the syringe unit 2 is attached and connected to the drive unit 3 to assemble the autoinjector 1 as described herein. FIG. 2 depicts a perspective view of the pre-assembled syringe unit 2 separated from the pre-assembled drive unit 3.

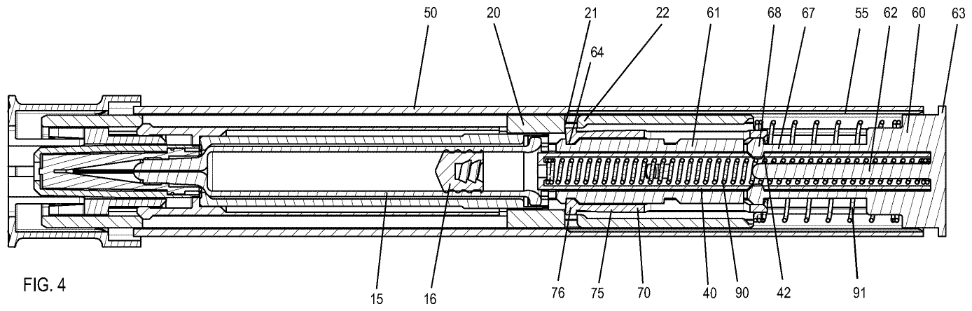

The pre-assembled syringe unit 2 as shown in FIG. 2 and FIG. 4 includes a sleeve-shaped housing 50, a needle cover in form of an elongated sleeve 20, a cap 10 mounted on a distal end, the syringe holder 30 adapted to hold the syringe 15 such that it cannot move relative to the housing and the prefilled syringe (PFS) 15 containing a liquid drug and a piston 16 which is movable inside a syringe barrel. A rigid needle shield (RNS) 17 is initially mounted on the injection needle 18. The housing, the needle cover sleeve, the cap and the syringe holder are injection-molded plastic parts for instance may be made of polypropylene (PP).

The drive unit 2 (FIGS. 1 and 4) includes a supporting member 60, a hollow plunger rod 40, an injection spring 90 to move the plunger rod 40 in the dispensing direction, a cover sleeve spring 91 adapted to bias the needle cover sleeve 20 in a covering position if the syringe unit 2 is connected to the drive unit 3 and a lock sleeve 70 arranged on an outside of a sleeve-shaped portion 61 of the supporting member 60. The supporting member, the plunger rod and the lock sleeve are injection-molded plastic parts and in particular may be made of polyoxymethylene (POM) or another technical plastic.

In the following, the assembly of the autoinjector 1 is described. Subsequently, the function and the injection with the autoinjector is described in detail.

In order to assemble the pre-assembled syringe unit 2, the needle cover sleeve 20 is inserted into the housing 50. As the housing 50 has a rectangular cross-section the needle cover sleeve 20 cannot rotate relative to the housing but is movable along the longitudinal axis. Then, the cap 10 is mounted on a distal end of the needle cover sleeve 20.

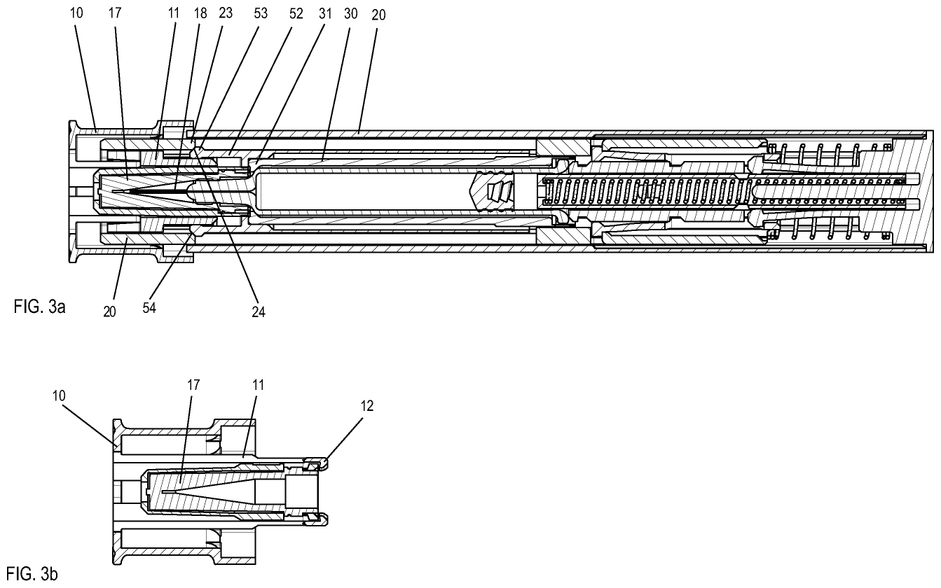

FIG. 3a shows a sectional view of the autoinjector with the cap mounted onto the distal end of the needle cover sleeve 20. FIG. 3b shows a section view through the cap 10 when it is separated from the autoinjector. The plane for the sectional view in FIG. 3b is 90° rotated compared to the plane for the sectional view of FIG. 3a.

As shown in FIGS. 3a and 3b the cap 10 includes inside a RNS remover sleeve 11 extending along the longitudinal axis and fixedly connected to an outer portion of the cap. The remover sleeve 11 includes a rim with hooks 12 (FIG. 3b) adapted to engage a proximal end of the RNS 17. If the cap 10 is mounted as shown in FIG. 3a, the remover sleeve 11 is radially between the RNS 17 and securing arms 52 of the housing 50. The securing arms 52 have free ends including knob-shaped ends 53. The securing arms 52 are arranged radially between an inner side of the needle cover sleeve 20 and an outside surface of the remover sleeve 11. The needle cover sleeve 20 includes securing ribs 23 extending along the longitudinal axis and including proximally faced sloped surfaces 24 that engage corresponding counter sloped surfaces 54 on the knob ends 53 of the securing arms 52. As the surfaces 24 engage the counter surfaces 54 and as the securing arms 52 are held in place by the remover sleeve 11, the needle cover sleeve 20 is prevented from being moved proximally.

As a next assembly step, the syringe holder 30 is inserted from a proximal side into the housing 50 but is not yet moved into a distal end position. The housing 50 includes a connecting portion 55 (see FIG. 4) extending proximally of the inserted syringe holder and having a length of about the axial length of the syringe holder 30. This connecting portion 55 accommodates the supporting member 60 when the syringe unit is connected to the drive unit.

After the above assembly step, the syringe unit 2 is in its pre-assembled state and all components of the syringe unit are fixed and cannot dethatch or be separated unless a (snap-fit) connection is released.

At the drug manufacturer site or at the assembly site, the prefilled syringe 15 with its RNS 17 is inserted from the proximal side into the syringe holder 30 and deflects a distal rim 31 (FIG. 3a) of the syringe holder 30 to let the RNS 17 pass. The rim 31 then returns back into its original position and thus the rim 31 is located between the proximal end of the RNS 17 and a distal shoulder of the syringe barrel. The syringe holder 30 together with the syringe 12 is then moved into its final distal position within the housing 50 until the holder abuts a stop rib of the housing 50 as shown in FIG. 3a. In this final position, the rim 31 is prevented from being deflected or moved radially outwards by the housing, which encounters the rim 31 and holds the rim in place.

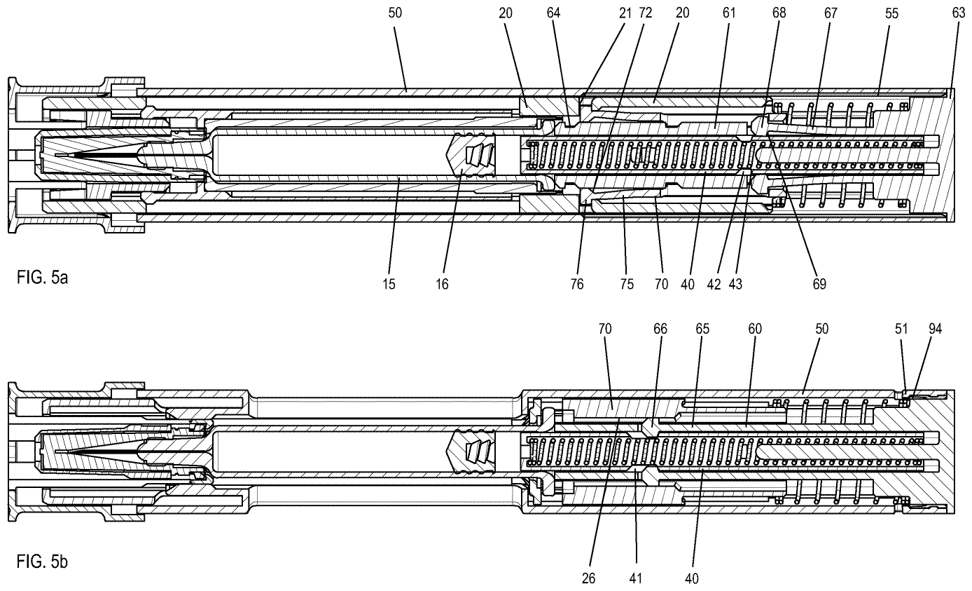

For the pre-assembly of the drive unit 3, the injection spring 90 in form of a spiral spring is inserted into a bore or opening of the sleeve-shaped portion 61 of the supporting member 60 such that the windings of the spring 90 are located radially between an inner guiding pin 62 and the inner wall of the sleeve-shaped portion 61 as shown in FIG. 4. Subsequently, the plunger rod 40 is inserted with its proximal end into the sleeve-shaped portion 61 until knobs 66 at the end of distal retaining arms 65 snap into axially elongated distal recesses 41 in the plunger rod (see FIG. 5b). Knobs 66 slide distally inside the recesses 41 until knobs 68 of proximal retaining arms 67 snap into proximal recesses 42 in a side wall of the plunger rod (shown in FIG. 4 and in released state in FIG. 5a). The two distal retaining arms 65 are oppositely arranged to each other and the two proximal retaining arms 67 are oppositely arranged to each other. Moreover, the two pairs of retaining arms 65, 67 are 90° rotated relative to each other. A perspective view of the supporting member 60 with the distal and proximal retaining arms 65, 67 with their respective knobs 67,68 can be seen in FIG. 1. After this assembly step, the injection spring 90 in the compressed state is exclusively held in place by the proximal retaining arms 67, as the knobs 66 of the distal retaining arms no longer touch the proximal end surface of the distal recesses 41.

Then, the cover sleeve spring 91 is mounted to the sleeve shaped portion 61 and shifted in the proximal direction until the proximal end of the cover sleeve spring 91 abuts a support surface 96 on a supporting rib shown in FIG. 1. As a final assembly step, the lock sleeve 70 is mounted onto the sleeve-shaped portion 61 and moved towards a proximal end wall 63 until cam ends 76 of deflectable locking arms 75 of the lock sleeve 70 snap into distal recesses 64 in the supporting member 60 (as shown in FIG. 4) such that the lock sleeve 70 is held onto the supporting member by a snap-fit connection.

The drive unit 3 is then in its pre-assembled state as shown in FIG. 2. That means all components of the pre-assembled drive unit 3 are fixed and cannot be detached or separated unless a snap-fit connection is released. That is, the cover sleeve spring 91 is compressed and held in place by a ledge 71 of the lock sleeve and the latter is held onto the supporting member by its locking arms 75. The injection spring 90 is compressed and biases the plunger rod 40 in the distal dispensing direction. The plunger rod 40 is held in place by the proximal retaining arms 67 and the arms 67 in turn are held in their holding position by the lock sleeve 70 as an inner surface of the lock sleeve abuts the knobs 68 and thus prevents the proximal retaining arms 67 from being deflected radially outwards.

As a last assembly step for assembling the autoinjector, the pre-assembled syringe unit 2 with the inserted syringe is connected to the pre-assembled drive unit 3. For that purpose, the sleeve-shaped portion 61 is inserted into the connecting portion 55 of housing 50 from a proximal side. FIG. 4 depicts a state where the drive unit 3 is partly inserted but not yet completely connected to the syringe unit 2.

As a proximal end of the needle cover sleeve 20 is located proximally to the ledge 71 (but rotated by 90° around the longitudinal axis) the cover sleeve spring 91 directly encounters the needle cover sleeve 20. That means the cover sleeve spring 91 is held in its compressed state exclusively by the needle cover sleeve 20 and thus biases the cover sleeve in its covering position when the drive unit is connected to the syringe unit.

As shown in FIG. 4, during assembly the locking arms 75 encounter a ledge 21 inside the needle cover sleeve 20. When the drive unit 3 is further moved distally into the housing 50, the lock sleeve 70 is stopped and does not move any further as cam ends 76 engage with the ledges 21. That means the supporting member 60 together with the plunger rod 40 and the injection spring are moved relative to the lock sleeve 70 and hence relative to the housing 50. That in turn causes the locking arms 75 to deflect radially outwards as the arms include sloped surfaces 72 (FIG. 5a) on their free ends and as the cam ends 76 are pushed against the ledges 21. The cam ends 76 are thereby locked to the cover sleeve 20 as each cam end 76 is held in a gap between the ledge 21 and a distal snap element 22 of the needle cover 20.

Furthermore, as the knobs 68 of the proximal retaining arms 67 include a proximally oriented sloped surface 69 engaging a distally oriented sloped surface 43 in proximal recess 42 of the biased plunger rod 40 (see FIG. 5a), the retaining arms 67 are allowed to deflected radially outwards as the injection spring moves the plunger rod 40 a short distance and hence the sloped surfaces 69, 43 slide along each other pressing the retaining arms 67 radially outwards. This in turn means the plunger rod 40 is released from the proximal retaining arms 67. The plunger rod 40 is then exclusively held in place by the distal retaining arms 65 engaging the distal recesses 41 in the plunger rod (see FIG. 5b).

FIGS. 5a and 5b show a fully assembled autoinjector 1 where the drive unit 3 is completely inserted into the syringe unit 2 and connected thereto. As shown, the supporting member 60 is completely inserted into the connecting portion 55 such that a proximal end wall 63 closes the proximal opening of the housing 50. A distal end of the supporting member 60 abuts a flange portion of the syringe 15 and thereby fixates the syringe 15 inside the autoinjector. As shown in FIG. 5a the proximal retaining arms 67 are deflected radially outwards.

FIG. 5b shows the same state of the autoinjector 1 as FIG. 5a but the cut for the sectional view is in a plane 90° rotated compared to the plane of the sectional view of FIG. 5a. As shown in FIG. 5b a proximal end portion of the supporting member 60 is connected to the proximal end of the housing by a snap element 51 in the housing 50 engaging a protrusion 94 of the supporting member 60. The supporting member 60 is thus fixed to the housing 50 and cannot move relative thereto.

In FIG. 5b it can be seen that the knobs 66 of the distal retaining arms 65 are within the distal recesses 41 of the plunger rod 40 and thus hold the plunger rod in place. The distal retaining arms 65 are prevented from being deflected outwards by an inner surface 26 of the needle cover sleeve 20.

In order to prepare the autoinjector for an injection, the user removes the cap 10. As the remover sleeve 11 of the cap engages the RNS 17 with its hooks 12 (see FIG. 3b) the RNS is removed together with the cap 10 when the cap is pulled off from the autoinjector.

When the cap 10 is removed from the needle cover sleeve 20, the securing arms 52 of the housing 50 are no longer prevented from being deflected radially inwards. That means if a force acts on the needle cover sleeve in the proximal direction the needle cover sleeve 20 can move proximally thereby deflecting the knob ends 53 and the securing arms 52 can deflect radially inwards as the sloped surfaces 24, 54 slide along each other.

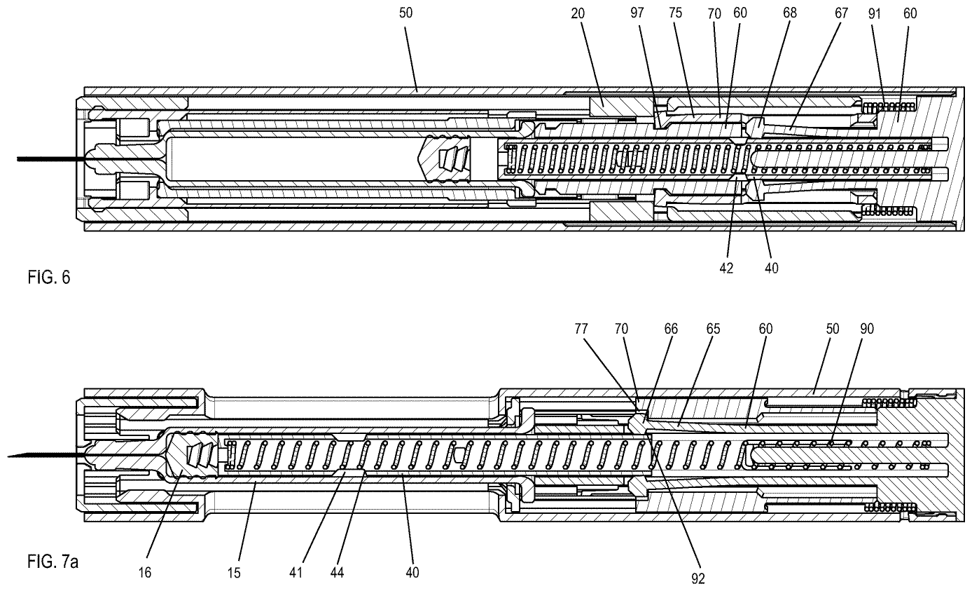

To start the injection, the user presses a distal end of the needle cover sleeve 20 onto the injection site and thereby pushes the needle cover sleeve 20 into the housing against the spring force of the cover sleeve spring 91. The cover sleeve 20 thus moves from its covering position into its retracted position (see FIG. 6). This movement triggers several functions.

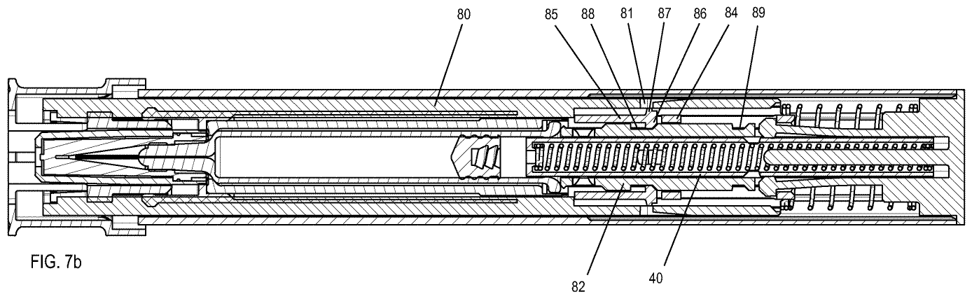

Firstly, the proximal movement of the needle cover sleeve 20 also moves the lock sleeve 70 in a proximal end position as the lock sleeve is axially connected to the needle cover sleeve, shown in FIG. 6. This causes the distal retaining arms 65 to get out of engagement with the inner surface of the lock sleeve 70. As the knobs 66 of arms 65 have proximally oriented sloped release surfaces 92 sliding against corresponding distally oriented release sloped surfaces 44 of the distal recess 41 of the plunger rod (FIG. 7a), the knobs 66 are forced radially outwards into a distal recess 77 in the lock sleeve 70 as shown in FIG. 7a. Therefore, the distal retaining arms 65 deflect radially outwards and thus release the plunger rod 40. That means the compressed injection spring 90 can release and thus move the plunger rod 40 in the dispensing direction to move the piston 16 inside the syringe barrel to dispense the drug from the syringe 15. The plunger rod is axially guided by the sleeve-shaped portion 61 which has a rectangular cross-section with rounded corners 95 (see FIG. 1) corresponding to a plunger rod proximal end flange. The plunger rod 40 is thus prevented from being rotated.

Secondly, the radially deflected distal retaining arms 65 further hold and lock the lock sleeve 70 in the proximal end position such that the lock sleeve and cannot move again in the distal direction.

Thirdly, as shown in FIG. 6, as the lock sleeve 70 is no longer pushed distally against the ledge 21, the locking arms 75 can ultimately deflect back radially inwards into a proximal recess 97 in the supporting member meaning that the axial connection between the needle cover sleeve 20 and the lock sleeve 70 is disengaged.

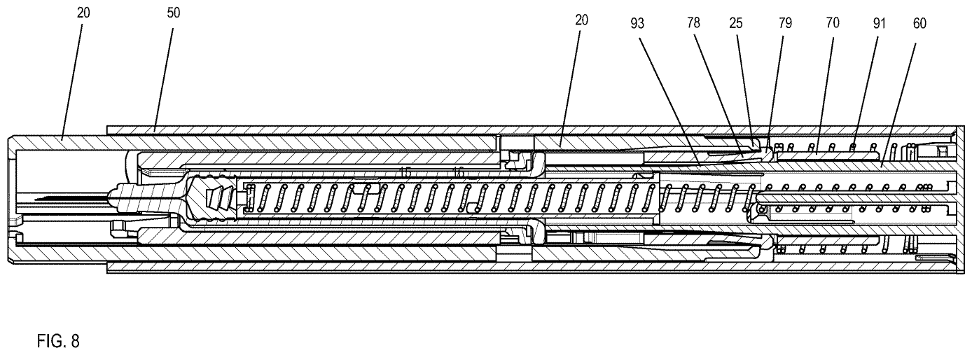

Fourthly, the proximal holding arms 78 with holding cams 79 of the lock sleeve 70 can deflect radially outwards by means of a ramp 93 (FIG. 8, whose section plane is rotated by 45° relative to the other FIGS.) on an outside of the sleeve-shaped portion 61. As shown in FIG. 8 and as described hereafter, the holding cams 79 can engage the needle cover sleeve 20 to lock it axially.

Once the injection is completed the user removes the autoinjector 1 from the injection site. Due to the cover sleeve spring 91, the needle cover sleeve 20 is moved from the retracted position back into its distal covering position. This state of the autoinjector 1 is shown in FIG. 8. Shortly before the needle cover sleeve 20 reaches its final covering position, proximal snap elements 25 of the needle cover sleeve 20 pass the lock sleeve holding cams 79. As the snap elements include an angled surface on a distal side but a stop surface on a proximal side, the needle cover sleeve 20 can move distally but is prevented from being moved back in the proximal direction once it has passed the deflected holding cams 79. The needle cover sleeve 20 is thus locked in its covering position (needle cover lock).

After use the autoinjector is configured to be disassembled and the syringe unit plastic parts made of PP can be discarded separately from the drive unit plastic parts made of POM.

Alternatively, the drive unit and the syringe unit without the syringe is configured to be reused. In this case, at a manufacturer site or reprocessing site, a skilled person carries out a reset procedure to prepare the autoinjector for reissue to consumers.

Before disassembly, the autoinjector may be logged either by means of an electronic tag such as an NFC tag or by a serial number. If the lifetime is not yet exceeded, the autoinjector is forwarded for refurbishment.

In a first reset step, a specific disassembly tool is inserted next to the housing snap elements 51 to release the snap-fit connection between the housing 50 and the supporting member 60. Thus, the syringe unit 2 and drive unit 3 are configured to be separated. The emptied syringe 15 is then removed from the syringe unit and disposed of.

In a next step, the lock sleeve 70 is disassembled from the supporting element 60 and the plunger rod 40 is shifted from a distal end position back into a proximal end position and thereby the injection spring 90 is biased again. The lock sleeve 70 is then mounted to the supporting element again and the plunger rod 40 is held in place by the proximal retaining arms 67 as described above.

In a further step, the injection spring force may be tested by releasing the plunger rod 40 to determine whether the injection spring 90 fulfills the requirements for a reissue. If the spring force does not reach a predefined threshold or if any malfunction of the drive unit 3 is detected, the drive unit 3 is discarded and replaced by a new drive unit.

In a further step, the syringe unit 2 and the drive unit 3 are subject to an industrial cleaning or sterilization. Further, a new prefilled syringe 15 is inserted into the syringe holder 30. The syringe unit and the drive unit can then be connected again as described above to assemble the autoinjector for reissue and reuse.

FIG. 7b depicts a further version of the first embodiment with a different lock sleeve 84 and a slightly different supporting member 82. In contrast to the lock sleeve previously described the lock sleeve 84 shown in FIG. 7b has proximally extending locking arms 85 each having a cam 86 on a proximal end. The needle cover sleeve 80 has a slightly modified recess 81 compared to the needle cover sleeve 20 previously described.

FIG. 7b shows the state of the autoinjector when the drive unit is connected to the syringe unit and the plunger rod is held in place by distal retaining arms. However, in the state shown in FIG. 7b, the cam ends 86 of the locking arms 85 have not yet inserted into recess 81 in the cover sleeve 80 to axially couple the lock sleeve 84 to the cover sleeve. The distal recess 88 of the supporting member 82 includes a sloped proximal end wall and the cam ends 86 include a corresponding sloped wall causing the locking arms to deflect radially outwards. Each cam end 86 additionally includes a protrusion 87 adapted to engage the recess 81 to facilitate coupling to the needle cover sleeve 80.

The supporting member 82 further includes a second proximal recess 89 to accommodate the cam ends 86 after dispensing when the lock sleeve is in its distal end position as previously described. All other functions are the same as previously described with respect to the first version of the first embodiment.

### Second Embodiment

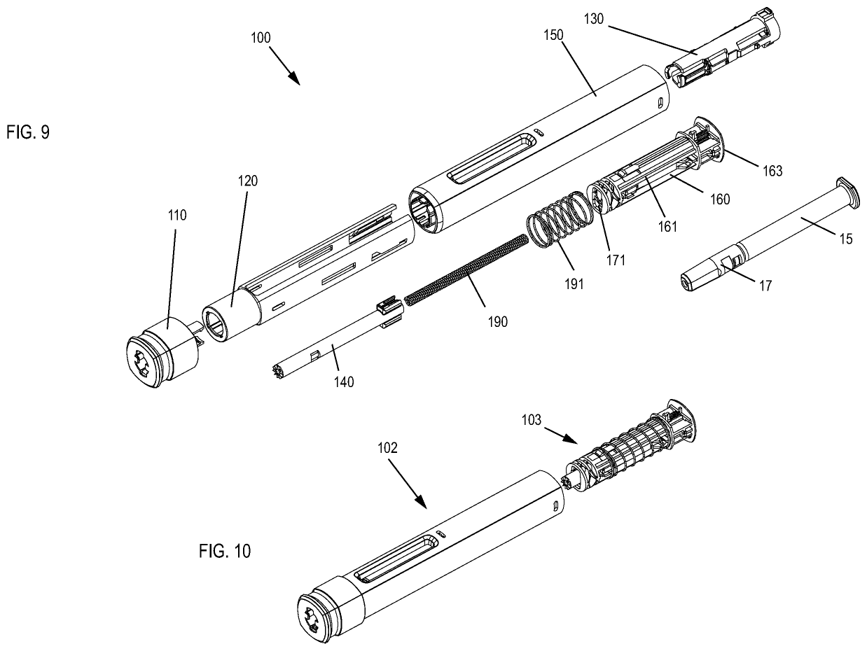

FIG. 9 depicts a perspective exploded view of an injection device according to a second embodiment of the present disclosure in form of an autoinjector 100. The autoinjector 100 includes a pre-assembled syringe unit 102 and a pre-assembled drive unit 103 (see FIG. 10). During assembly, a prefilled syringe 15 is inserted into a syringe holder 130 and the syringe unit 102 is attached and connected to the drive unit 103 to assemble the autoinjector 100. FIG. 10 depicts a perspective view of the pre-assembled syringe unit 102 separated from the pre-assembled drive unit 103 according to the second embodiment.

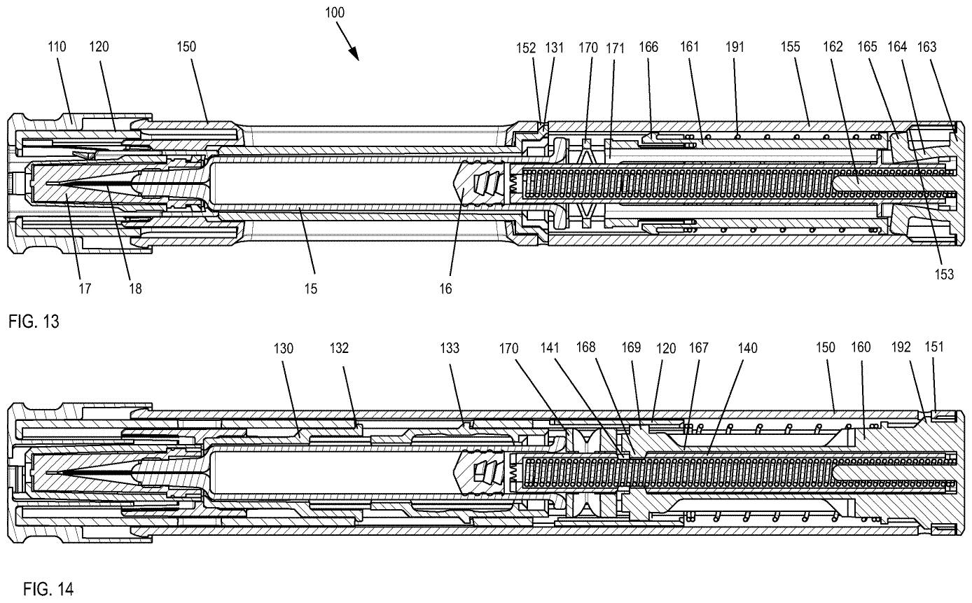

The syringe unit 102 as best shown in FIGS. 9 and 13 includes a sleeve-shaped housing 150, a needle cover sleeve 120, a cap 110, the syringe holder 130 adapted to hold the syringe 15 such that it cannot move relative to the housing and the prefilled syringe (PFS) 15 containing a liquid drug and a piston 16. A rigid needle shield (RNS) 17 is initially mounted on the injection needle 18. The housing 150, the needle cover sleeve 120, the cap 110 and the syringe holder 130 are injection-molded plastic parts and all made of polypropylene (PP).

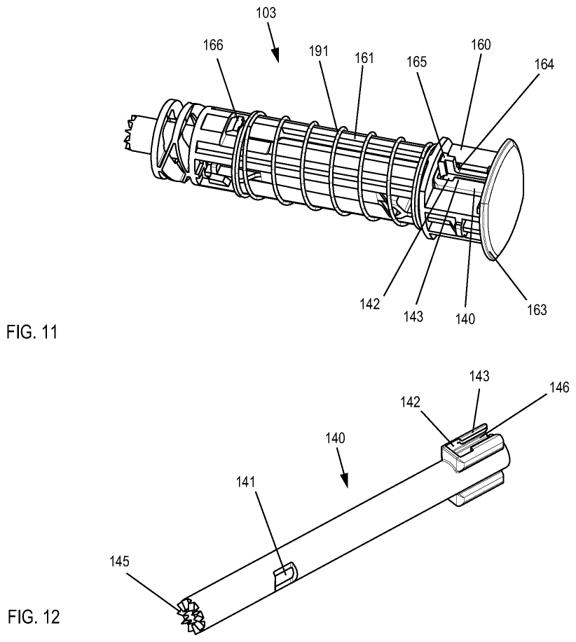

The drive unit 102 shown in FIGS. 9 and 11 includes a supporting member 160, a hollow plunger rod 140, an injection spring 190 to move the plunger rod 140 in the dispensing direction, and a cover sleeve spring 191 adapted to bias the needle cover sleeve 120 in a covering position if the syringe unit 102 is connected to the drive unit 103. The supporting member 160 and the plunger rod 140 are injection-molded plastic parts and may in particular be made of polyoxymethylene (POM).

In order to assemble the syringe unit 102, the needle cover sleeve 120 is inserted into the housing 150 from a proximal side. The housing 150 has a rectangular cross section which prevents the needle cover sleeve 120 to rotate relative to the housing but the cover sleeve is movable along the longitudinal axis relative to the housing 150. Then, the cap 110 is mounted on a distal end of the needle cover sleeve 120. As a next assembly step, the syringe holder 130 is inserted from a proximal side into the housing 150 but it is not yet moved into a final distal position.

After the above assembly step, the syringe unit 102 is in its pre-assembled state and all components of the syringe unit are fixed and cannot be detached or separated unless a snap-fit connection is disengaged.

At the drug manufacturer site or at the assembly site, the prefilled syringe 15 with its RNS 17 is inserted from the proximal side into the syringe holder 130. The syringe holder 130 together with the syringe 112 is then moved into its final distal position within the housing 150 until a proximal flange 131 of the holder 130 snaps into a recess 152 in the housing 150 to establish a snap-fit connection, see FIG. 13. Furthermore, the syringe holder 130 includes in a middle portion and in a proximal portion two axially distant noses 132, 133 that snap into a recess in the needle cover sleeve 120 to control proximal and inhibit distal movement of the latter as shown in FIG. 14.

FIG. 11 shows a perspective view of the pre-assembled drive unit 103 and FIG. 12 depicts a perspective view of the plunger rod 140 alone. For the pre-assembly of the drive unit 103 the injection spring 190 in form of a spiral spring is inserted into a bore or opening 171 of a sleeve-shaped portion 161 of the supporting member 160 (FIG. 9 or 13) such that the windings of the spring 190 are located radially between an inner guiding pin 162 and the inner wall of the sleeve-shaped portion 161 (see FIG. 13). Subsequently, the hollow plunger rod 140 is inserted with its proximal end into the opening 171 until the plunger rod snaps into the supporting member. That is, the plunger rod 140 includes two oppositely arranged connecting portions 146 shown in FIG. 12. Each connection portion includes two axially oriented holding ribs 143 and a recess 142 arranged on a distal end of the ribs 143 as best shown in FIG. 12. The supporting member 160 includes in a proximal portion two oppositely arranged deflectable L-shaped snap elements 164 (see FIG. 11) adapted to snap into the recess 142. The L-shaped snap elements 164 include a radially outwards protruding cam 165.

As shown in FIG. 11, the snap element 164 is positioned in the recess 142 distally to the holding ribs 143 and held in place by the holding ribs 143. As the injection spring 190 is compressed, the plunger rod 140 is biased in the distal direction and held in place by the snap elements 164 such that the plunger 140 rod cannot move relative to the supporting member 160. As shown in FIG. 12 the plunger rod 140 includes on its distal end arrowhead-shaped protrusions 145 extending in the distal direction and adapted to engage and enter in the syringe piston made of rubber.

As a next assembly step, the cover sleeve spring 191 is mounted to envelop the sleeve-shaped portion 161. The cover sleeve spring 191 is compressed and held in place by a radially protruding snap element 166 located in the sleeve-shaped portion 161, see FIG. 11.

The drive unit 103, as shown in FIGS. 10 and 11, is then in its pre-assembled state. All components of the drive unit 103 are fixed and cannot be detached or separated unless a snap-fit connection is disengaged.

To assemble the autoinjector 100, the pre-assembled syringe unit 102 with the inserted syringe is connected to the pre-assembled drive unit 103. That is, the sleeve-shaped portion 161 is inserted into a connecting portion 155 of the housing 150 from a proximal side as shown in FIG. 13.

FIG. 13 depicts a sectional view of the completely assembled autoinjector 100 according to the second embodiment. In the assembled state, the drive unit 103 is completely inserted and connected to the syringe unit 102. During insertion, the cover sleeve spring 191 is released from the snap element 166 and encounters a proximal end of the needle cover sleeve 120. Therefore, the cover sleeve spring 191 biases the cover sleeve 120 in the distal direction in a covering position.

Moreover, as shown in FIG. 13, during insertion the L-shaped snap elements 164 are deflected radially inwards. That is, because the radial cams 165 of the snap elements 164 encounter sloped contact surfaces 153 inside the proximal housing the cams 165 on the L-shaped snap elements 164 are forced inwards. The deflection of the snap elements 164 in turn releases the snap-fit to the plunger rod 140 and thus the spring-biased plunger rod moves a short distance in the distal direction until radially inwardly extending protrusions 168 of two oppositely arranged retaining arms 167 engage proximal ends of oppositely arranged recesses 141 in the plunger rod 140.

The retaining arms 167 are shown in FIG. 14, which depicts the same state of the autoinjector 100 as FIG. 13 but the plane of the sectional view is 90° rotated compared to the plane of the sectional view of FIG. 13. Each radial protrusion 168 further includes a radially outward extending guiding cam 169, which abuts an inner surface of the needle cover sleeve 120. The retaining arms 167 are thus prevented from being deflected radially outwards and thus the plunger rod 140 is held in place and locked by the retaining arms 167.

Furthermore, the supporting member 160 includes on its distal end a spring element 170, which is monolithic with the sleeve-shaped portion 161 and thus integrally formed in the sleeve-shaped portion. When the drive unit 103 is connected to the syringe unit 102, a distal end of the spring element 170 encounters a proximal end of the syringe 15 and thus biases the syringe 15 in the distal direction against a distal stop surface inside the syringe holder 130.

A proximal end portion of the supporting member 160 is connected to the proximal end of the housing 150 by a snap element 151 in the housing 150 (FIG. 14) engaging a nose 192 of the supporting member. The supporting member 160 is thus fixed to the housing 150 and cannot move relative thereto.

To start the injection, the user removes the cap 110 and presses a distal end of the needle cover sleeve 120 onto the injection site. Thereby, the needle cover sleeve 120 is pushed into the housing 150 against the spring force of the cover sleeve spring 191. The cover sleeve 120 is thus moved from its covering position into its retracted position (push-on-skin).

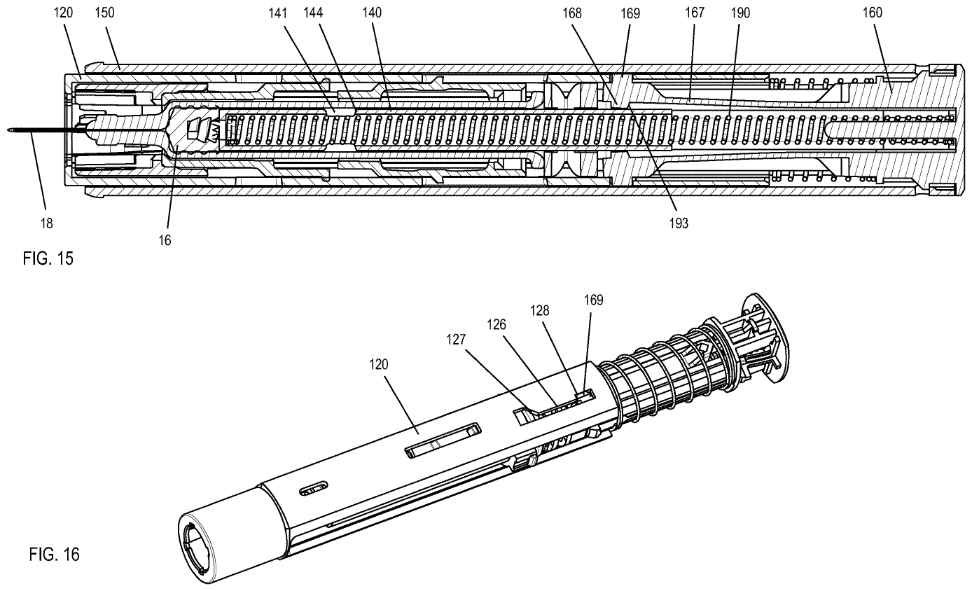

FIG. 15 depicts a state when the cover sleeve 120 is in the retracted position and the autoinjector has completely injected the liquid drug.

When the needle cover sleeve 120 is in the retracted proximal end position, each guiding cam 169 of the retaining arms 167 gets axially aligned with a recess or hole in the cover sleeve 120, which forms a distal leg of a U-shaped channel 126 in the needle cover sleeve 120. One of two oppositely arranged channels 126 is depicted in FIG. 16, which shows a perspective view of the needle cover sleeve and the drive unit. Other components and in particular the housing of the autoinjector is not depicted in FIG. 16.

As can be seen in FIG. 15, the two oppositely arranged recesses 141 in the plunger rod each have a sloped proximal end surface 144 and, as the protrusions 168 have corresponding sloped surfaces 193, the protrusions 168 are forced under the bias of the injection spring 190 to move radially outwards and hence the retaining arms 167 deflect radially outwards. Thereby, the guiding cams 169 enter the distal leg of the U-shaped channels 126.

Due to the radially outwardly deflected retaining arms 167, the plunger rod is released and thus the injection spring 190 can release and moves the plunger rod 140 in the distal direction. The piston 16 inside the syringe barrel is then moved until reaching a distal end position, as shown in FIG. 15. Upon contact of the plunger rod distal end with the piston 16, the arrowheads 145 of the plunger rod tip enter the rubber of the piston, thereby damping the impact of the plunger rod to the piston to avoid damage of the syringe barrel.

When the user removes the autoinjector 100, the needle cover sleeve 120 is moved from the retracted position back into its distal covering position due to the spring force of the cover sleeve spring 191.

The guiding cams 169 inside the distal legs of the U-shaped channels 126 can move out of the distal area, as a proximal side of the cam 169 as well as corresponding angled wall 127 in the channel 126, as shown in FIG. 16, are sloped with respect to the longitudinal axis. Therefore, as the guiding cam 169 can travel within the channel 126 and the needle cover sleeve 120 can move distally back in its covering position. Due to the U-shape of the channels 126, the guiding cams 169 and the retaining arms 167 are deflected tangentially during this movement. That means the retaining arms 167 are moved out of a plane running through the device center longitudinal axis and are deflected in a plane parallel and offset relative to the central longitudinal axis.

When the needle cover sleeve 120 has reached its covering position again and thus the distal end position, the guiding cams 169 flex tangentially into a proximal locking position in alignment with a stop wall 128. This state is shown in FIG. 16. If a force acts on the needle cover sleeve 120 in the proximal direction (towards the retracted position), e.g., when the device is pushed again onto a surface, the needle cover sleeve 120 is prevented from moving due to the guiding cams 169 encountering the stop walls 128 and cannot exit the locked proximal position inside the channels 126. This is because the stop walls 128 prevent any deflection or lateral movement of the guiding cams 169 (needle cover lock).

Similar to the first embodiment the autoinjector, the second embodiment is configured to be disassembled and the parts made of the same material can be discarded together.

Alternatively, and as previously described, the autoinjector may be reused after a reset procedure. Namely, the snap-fit connection between the supporting member 160 and the housing 150 are configured to be released to separate the pre-assembled syringe unit 102 from the pre-assembled drive unit 103. The plunger rod 140 is configured to be pushed back into a proximal end position to compress the injection spring 190. The plunger rod 140 can then be held in place by the L-shaped snap elements 164 as described above. Subsequently, a new syringe can be inserted in the syringe holder and the syringe unit can be reconnected to the drive unit.

### Third Embodiment

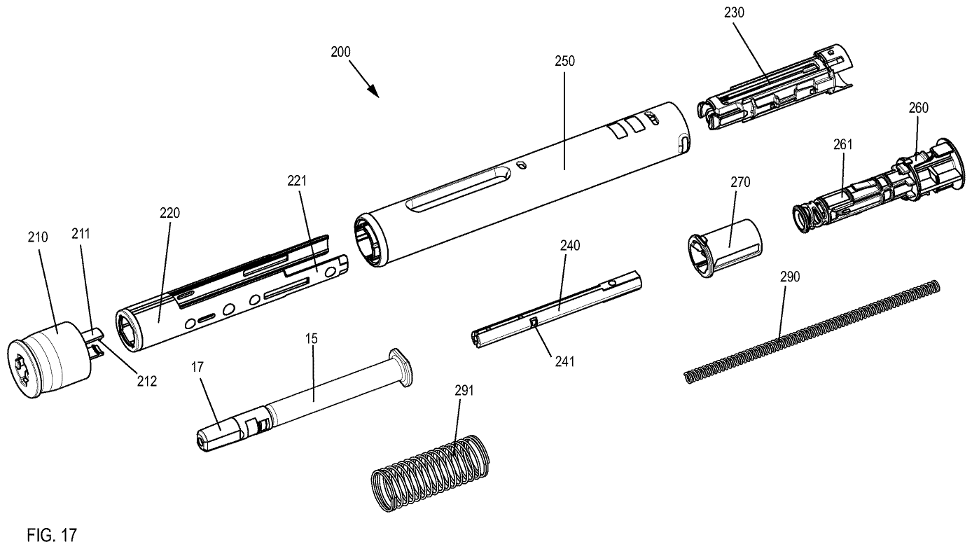

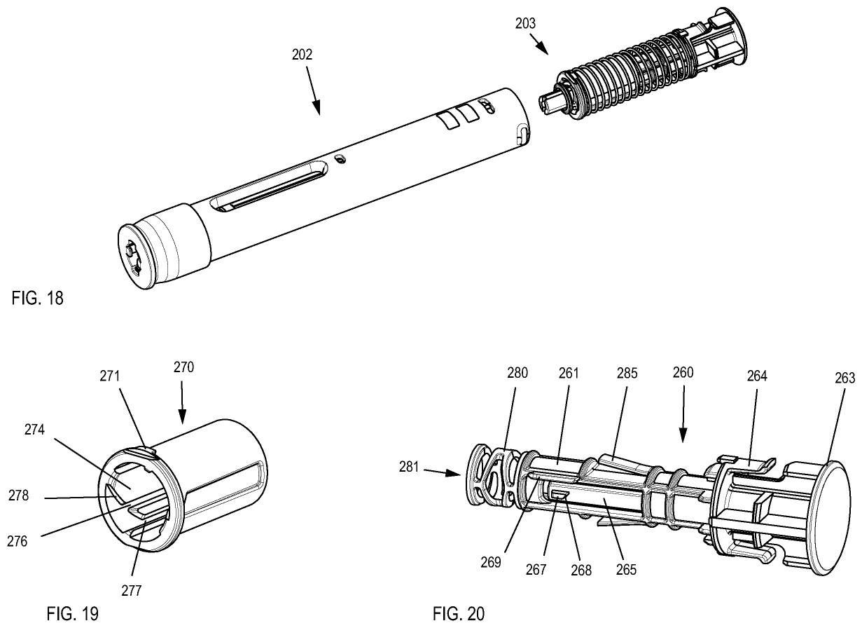

FIG. 17 depicts a perspective exploded view of an injection device according to a third embodiment of the present disclosure in form of an autoinjector 200. The autoinjector 200 includes a pre-assembled syringe unit 202 and a pre-assembled drive unit 203. FIG. 18 depicts a perspective view of the pre-assembled syringe unit 202 separated from the pre-assembled drive unit 203.

The syringe unit 202 includes a sleeve-shaped housing 250, a needle cover sleeve 220, a cap 210, a syringe holder 230 adapted to hold a syringe such that it cannot move relative to the housing and the prefilled syringe (PFS) 15 containing a liquid drug and a piston 16, which is movable inside a syringe barrel. A rigid needle shield (RNS) 17 is initially mounted on an injection needle 18. The housing, the needle cover sleeve, the cap and the syringe holder are injection-molded plastic parts for instance made of polypropylene (PP).

The drive unit 202 includes a supporting member 260, a hollow plunger rod 240, an injection spring 290, a cover sleeve spring 291 and a lock sleeve 270 arranged on an outside of a sleeve-shaped portion 261 of the supporting member 260. The supporting member, the plunger rod and the lock sleeve are injection-molded plastic parts and in particular may be made of polyoxymethylene (POM).

For the assembly of the syringe unit 202, the needle cover sleeve 220 is inserted into the housing 250. The housing 250 includes within its interior, longitudinal guiding rails (not shown) to guide the needle cover sleeve and to prevent rotation. Then, the cap 210 is mounted on a distal end of the needle cover sleeve 220. The cap includes two oppositely arranged and longitudinally extending tongues 211 with hooks 212 at their ends, which can engage a proximal end of the RNS 17. The syringe holder 230 is then inserted from a proximal side into the housing 250. The syringe holder includes two oppositely arranged cams 231, which snaps into a recess 251 in the housing (see FIG. 22) to form a snap-fit connection and thus the syringe holder 230 cannot move relative to the housing 250. The syringe unit 203 is then in its pre-assembled state and all components of the syringe unit are fixed and cannot be separated.

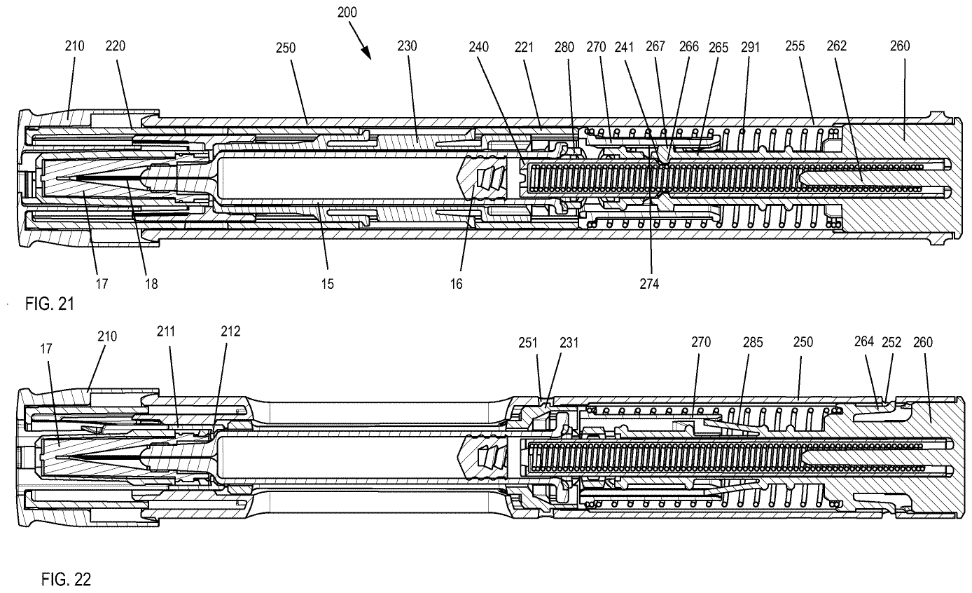

FIG. 20 depicts a perspective view of the supporting member 260. For the pre-assembly of the drive unit 203, the injection spring 290 in form of a spiral spring is inserted into a bore or opening 281 (FIG. 20) of the sleeve-shaped portion 261 of the supporting member 260 such that the windings of the spring 290 are located radially between an inner guiding pin 262 and the inner wall of the sleeve-shaped portion 261, shown in FIG. 21. Subsequently, the plunger rod 240 is inserted with its proximal end into the sleeve-shaped portion 261 until radially inwards extending protrusions 266 of retaining arms 265 snap into recesses 241 in the plunger rod (FIG. 21). The two retaining arms 265 are oppositely arranged to each other. Subsequently, the cover sleeve spring 291 is mounted onto the sleeve-shaped portion 261 and moved towards a proximal end wall 263.

FIG. 19 depicts a perspective view of the lock sleeve 270 alone. The lock sleeve 270 is mounted onto the sleeve-shaped portion 261. Details of the lock sleeve 270 are described below.

The drive unit 203 is then in its pre-assembled state. The plunger rod 240 is held in place by the retaining arms 265, which in turn are held in a holding position by the lock sleeve 270 as the inner surface of the lock sleeve abuts a radially outwards protruding cam 267 of the retaining arms 265 and thus prevents the retaining arms 265 from being deflected. The lock sleeve 270 includes a spring retaining nose 271 on a distal end to hold the biased cover sleeve spring in place.

As shown in FIG. 19 the lock sleeve 270 includes a first and a second axial guide in form of a first axial groove 276 and second axial groove 277, which are spaced apart from each other in a circumferential direction. The lock sleeve includes in total two oppositely arranged first grooves 276 and two second grooves 277. The first and second axial grooves 276, 277 extend along the longitudinal axis and are arranged on an inside of the lock sleeve. Furthermore, an angled guide section 278 is arranged on an inner side of the lock sleeve 270. Moreover, the lock sleeve 270 includes an engagement section with a locking surface 274, which protrudes radially inwards. The locking surface 274 is adapted to be in contact with cams 267 and holds the retaining arms 265 in their holding position.

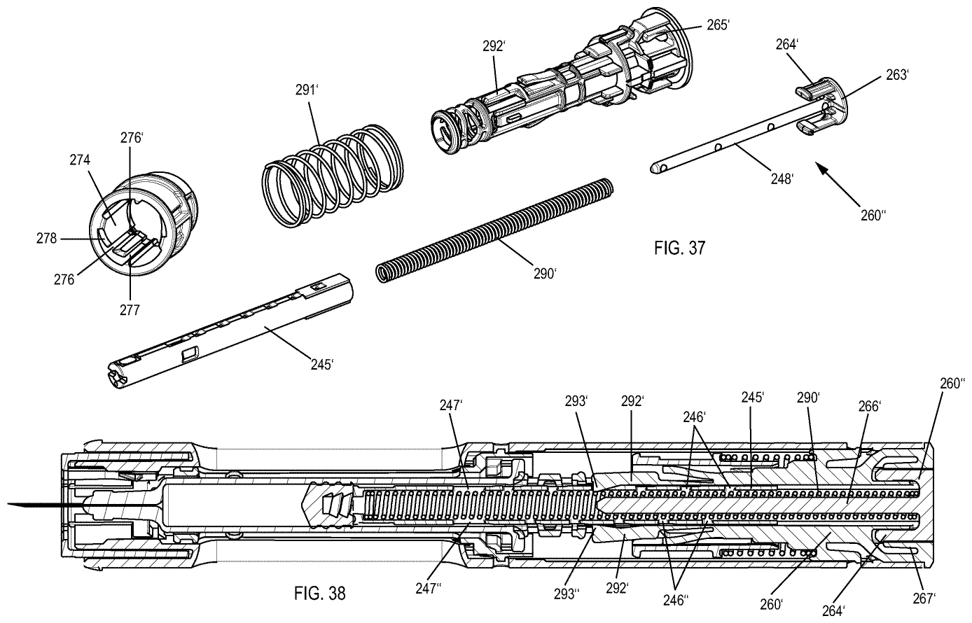

The supporting member 260 includes, on an outer surface of the sleeve-shaped portion 261, an axial rib 269 (FIG. 20) adapted to be inserted into the axials grooves 276, 277. The two axial grooves define a first and a second rotational position of the lock sleeve 270 relative to the supporting member 260 (and the housing). That means the axial rib 269 can either be in the first or in the second axial groove 276, 277 and thus the lock sleeve 270 is in its first or second rotational position. When the lock sleeve 270 is first mounted onto the supporting member 260, the axial rib 269 slides distally in the first axial groove 276 until it encounters a securing cam 276″ located in the first axial groove 276 (visible in FIG. 37). Securing cam 276″ has a sloped proximal wall that allows the axial rib 269 to pass in the distal direction, however it cannot move back proximally in groove 276 due to the non-sloped distal wall of cam 276″. This means that when sleeve 270 is mounted, once the axial rib 269 has passed securing cam 276″, the sleeve is fixedly mounted and cannot fall off (as long as axial rib 269 is located in first axial groove 276).

The supporting member 260 includes the radially outwardly protruding cams 267 on the free ends of the retaining arms 265. The cams 267 are adapted to be inserted into the first axial grooves 276 as described in detail below. As best shown in FIG. 20, each cam 267 has on its proximal side a wall 268 angled with respect to the longitudinal axis and adapted to cooperate with the angled guide section 278 of the lock sleeve 270.

The two axial grooves 276, 277 on each half of the locking sleeve are formed in a portion that protrudes radially towards a center of the lock sleeve. That means in a section where the grooves are formed, the lock sleeve has a smaller inner diameter. In a section spaced apart from the grooves, the lock sleeve has a larger inner diameter. This section with the larger inner diameter is adapted to accommodate locking arms 285 of the support member 260, which are best shown in FIG. 20. Moreover, the section with the larger inner diameter includes two oppositely arranged saw teeth 279 protruding radially inwards, shown in FIG. 24. On a distal side, each saw teeth 279 has a sloped surface 272 and on a proximal side the saw teeth includes a stop surface 273 adapted to engage a free end of the locking arm 285. However, the two locking arms 285 are only engageable with the saw teeth 279 when the lock sleeve 270 is in its second rotational orientation as described further herein.

As a last assembly step for assembly of the autoinjector, the pre-assembled syringe unit 202 with an inserted syringe 15 is connected to the pre-assembled drive unit 203. For that purpose, the sleeve-shaped portion 261 is inserted into a connecting portion 255 of the housing 250 from a proximal side.

FIGS. 21 and 22 depict the autoinjector in the assembled state where the drive unit 203 is completely inserted into the syringe unit housing 250 and connected thereto. FIG. 22 depicts the same state as FIG. 21 but the plane of the sectional view is 90° rotated compared to the plane of the sectional view of FIG. 21.

The supporting member 260 includes on its distal end a spring element 280, which is monolithic with the sleeve-shaped portion 261 and thus integrally formed in the sleeve-shaped portion. When the drive unit 203 is connected to the syringe unit 202, a distal end of the spring element 280 encounters a proximal end of the syringe 15 and thus biases the syringe 15 in the distal direction against a distal stop surface inside the syringe holder 230.

As can be seen in FIG. 22, a proximal end portion of the supporting member 260 is connected to the proximal end of the housing 250 by a snap arm 264 which engages a recess 252 in the housing such that the drive unit 203 is fixed to the housing 250 and cannot move relative thereto.

In FIG. 21, it can be seen that the cams 267 of the retaining arms 265 are held in place by the locking surface 274 of the lock sleeve 270, which prevents the retaining arms 265 from being deflected. The protrusions 266 are thus held in the recesses 241 (FIGS. 1 and 21) and therefore the biased plunger rod 240 is held in place.

To prepare the autoinjector 200 for an injection, the user has to remove the cap 210. The RNS 17 is removed together with the cap 10 as the tongues 211 with the hooks 212 engage the RNS (FIG. 17 and FIG. 22).

When the needle cover sleeve 220 is pressed onto the injection site, the needle cover sleeve 220 is pushed into the housing against the spring force of the cover sleeve spring 291. The cover sleeve 220 thus moves from its covering position into its retracted position.

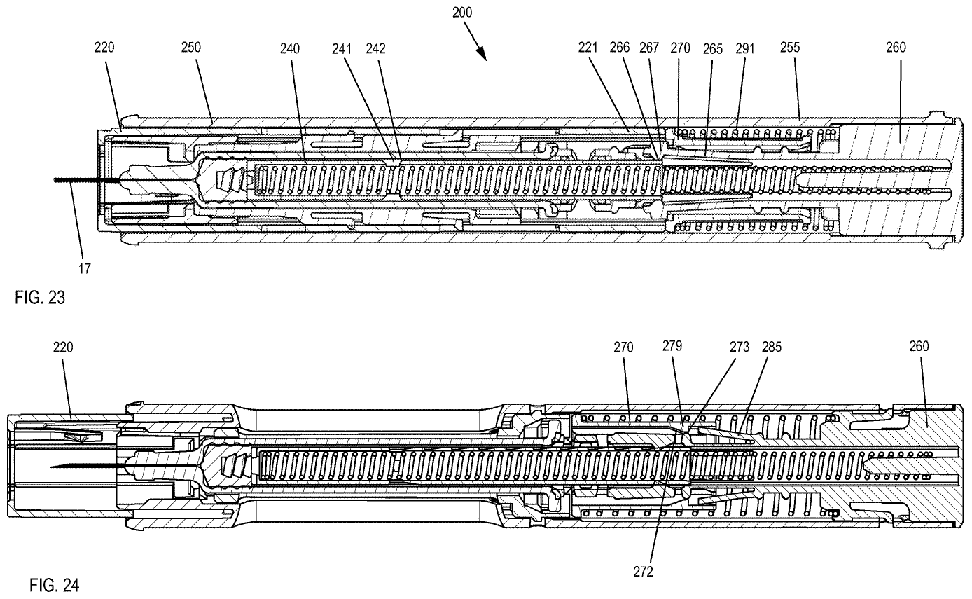

The proximal movement of the needle cover sleeve 220 also moves the lock sleeve 270 in a proximal end position as two extensions 221 of the needle cover sleeve encounter the lock sleeve 270, see FIG. 23. That means the locking surface 274 is shifted away from the cams 267 and the cams can get out of contact with the surface such that the retaining arms 265 are free to deflect radially outwards (not shown in FIG. 23). As the proximal end of the plunger rod recess 241 has a sloped surface 242 (FIG. 23), and as the protrusions 266 have corresponding proximal sloped surfaces, the surfaces slide along each other forcing the retaining arms 265 to deflect radially outwards due to the spring force of the compressed injection spring 290. That in turn means the plunger rod 240 is released and is moved in the dispensing direction by the injection spring 290 until the piston 16 reaches a distal end within the syringe barrel. This state is shown in FIG. 23. Although not shown in FIG. 23 the protrusions 266 are not in engagement with the plunger rod anymore and remain on an outside surface of the plunger rod once the plunger rod is released.

Shortly before the lock sleeve 270 reaches its proximal end position, the axial rib 269 of the supporting member 260 leaves the first axial groove 276 due to the proximally moved lock sleeve 270 and hence the lock sleeve is no longer axially guided and can be rotated relative to the supporting member.

When the injection is completed, the user removes the autoinjector 200 from the injection site. Due to the cover sleeve spring 291, the needle cover sleeve 220 is moved from its retracted position back towards its distal covering position. That means the lock sleeve 270 is no longer held in its proximal position and, as the axial rib 269 is out of engagement with first axial groove 276, the lock sleeve is free to rotate.

As the retaining arms 265 are deflected radially outwards, the angled walls 268 (shown in FIG. 20) of the cams 267 engage the angled guiding sections 278 (FIG. 19) of the lock sleeve. Due to the force of the compressed cover sleeve spring 291, the angled section 278 is pressed onto the angled wall 268, which forces the lock sleeve 270 to rotate counterclockwise (as seen in the distal direction) relative to the supporting member 260 (and thus relative to the housing) from its first rotational position unit the axial rib 269 hits a stop rib next to second axial groove 277, and hence the axial rib 269 is rotationally aligned with second axial groove 277. Coincidently, the cam 267 is rotationally aligned with the first axial groove 276. The lock sleeve is now in its second rotational position. As the cover sleeve spring 291 pushes the lock sleeve 270 in the distal direction, the axial rib 269 enters the second axial groove 277, and the cam 267 enters the first axial groove 276 thereby rotationally locking the lock sleeve 270 in its second rotational position.

As shown in FIG. 24, in the second rotational position the saw teeth 279 are rotationally aligned with the locking arms 285 of the supporting member 260. That means when the lock sleeve 270 is further moved distally the free ends of the locking arms 285 pass the sloped surface 272 of the saw teeth 279 and snap into the space proximal to the saw teeth stop surface 273. This state is shown in FIG. 24, which is a sectional view in a plane 90° rotated to the plane of the sectional view of FIG. 23. That means the locking arms 285 cannot pass the stop surface 273 and hence the lock sleeve 270 is prevented from being moved back in the distal direction. This in turn means the needle cover sleeve 220 cannot be moved towards its retracted position again and hence is locked in the covering position (needle cover lock).

Also, the autoinjector according to the third embodiment is configured to be disassembled and the parts made of the same material can be discarded together. Alternatively, as previously described with respect to the first embodiment, the autoinjector may be reused after a reset procedure.

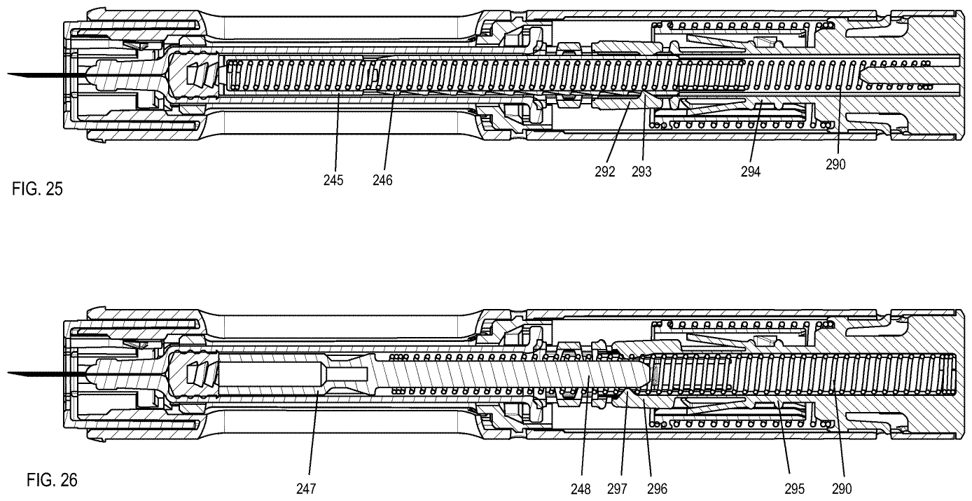

FIG. 25 depicts a further version of the third embodiment. In contrast the version of the third embodiment previously described the supporting member 294 of the autoinjector shown in FIG. 25 includes additionally two oppositely arranged and proximally directed, deflectable arms 292, each having a radially inwards extending nose 293. Furthermore, the plunger rod 245 includes two oppositely arranged flattened portions including nine saw teeth 246 arranged next to each other in the longitudinal direction. When the plunger rod 245 is released, and the injection spring 290 moves the plunger rod 245 in the dispensing direction and the noses 293 of the arms 292 slide over the saw teeth 246 thereby providing an audible and tactile feedback to the user during injection.

FIG. 26 depicts a further version of the third embodiment. In contrast to the autoinjector 200 of the third embodiment previously described, the version shown in FIG. 26 includes a supporting member 295 without a guiding pin. Instead, a guiding pin 248 is integrally formed in the plunger rod 247 as shown in FIG. 26. That means the plunger rod 247 does not have a hollow shape, which envelops the injection spring 290. The supporting member 295 includes two oppositely arranged and distally oriented deflectable arms 296, each having a nose 297 on their free end adapted to engage windings of the injection spring 290.

When the plunger rod 247 is released, the noses 297 of the arms 296 slide over the windings of the coil of the injection spring 290 and thus provide an audible and tactile feedback to the user during injection.

FIG. 37 depicts a fourth version of the third embodiment with an acoustic or tactile feedback mechanism alternative to the one shown in FIGS. 25 and 26. In this version, the supporting member 260′, 260″ is made up of two parts, a main body 260′ and a support body insert 260″. To assemble the drive unit in this version, the plunger rod is inserted from the proximal end into a bore of the main body 260′ of the supporting member and advanced in the distal direction until the noses 293′, 293″ of the elastic arms 292′ snap into leading recesses 247′, 247″ on each side of the plunger rod 240′ and protrusions on the inner side of snap arms 265′ snap into recesses 241′, the latter fixing the plunger rod in place. Subsequently, injection spring 290′ is mounted onto support pin 266′ of supporting member insert 260″ and the insert 260″ with the injection spring is inserted into the inner bore of supporting member main body 260′ until, upon maximum compression of the injection spring, the snap arms 264′ engage stop walls 267′, fixing the insert 260″ to the main body 260′. Finally, cover sleeve spring 291′ and lock sleeve 270′ are mounted on the supporting member and, upon final assembly, the drive unit is fixed to the syringe unit, as described with respect to other versions of this embodiment.

The acoustic feedback mechanism of this version, and as described next, is nevertheless independent of the way it is assembled, and applicable to all other embodiments, as well as generally to any kind of injection devices, in particular autoinjectors, with a plunger rod advancing a piston in the axial direction to dispense a liquid drug from a container through an injection needle.

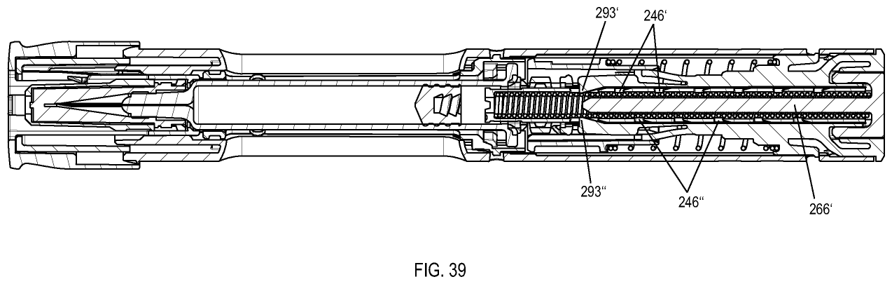

FIG. 38 and FIG. 39 depict the version of FIG. 37 in an intermediate state during injection, and in an initial state prior to injection, respectively. The plunger rod 240′ in this version includes six and five click recesses or cut-outs 246′, 246″ on two diametrically opposite sides, which are not aligned in the axial direction but alternatingly arranged on both sides. As the noses 293′, 293″ on the distally oriented, deformably mounted, elastic arms are axially aligned, during distal movement of the plunger rod 240′, the noses 293′, 293″ alternatively flex into the respective click recesses 246′, 246″, providing acoustic clicks and tactile feedback for the user. The alternating arrangement of the recesses provides for a total of eleven clicks as continuous feedback during injection. The alternating arrangement of the recesses increases the distance between successive recesses on one side as compared to nine or even eleven recesses on one side, which minimizes impact on structural integrity of the plunger rod 240′ and which increases the flexibility in the axial arrangement and design of the individual recesses. In particular, a deflecting surface of the recesses (oriented distally) and/or of the noses (oriented proximally) is sufficiently flat such that the force required for deflection and interference with the advancing movement of the piston is minimized. A slope angle of the deflecting surface with respect to the longitudinal axis may be less than 45°, such as less than 30°. This leads to an increased extension of the deflecting surface in the axial direction as compared to sloping angels beyond 45°, which is however perfectly compatible with a reduced number of recesses per side. Consistently, a total of 12 clicks for a 1 ml syringe has proven to be sufficient.

The mounting of plunger rod 240′ from the proximal end allows for generation of louder click sounds as the plunger rod only needs to be able to slide in the distal direction inside the supporting member, and thus the distal end walls of the recesses 246′ and/or the noses 293′ on the elastic arms 292′ need not be sloped and can be perpendicular to the longitudinal axis such as to prevent proximal movement of the plunger rod. This results in an instantaneous release of the previously biased nose 293′ and inwardly flexing movement during injection, a maximum radial acceleration distance, a higher impact energy and thus a louder click. Additionally, the elastic flexible arms 292′ are only bent once during mounting and can then relax in leading recesses 247′ until injection, preventing material fatigue and resulting in a higher tension during injection.

### Fourth Embodiment

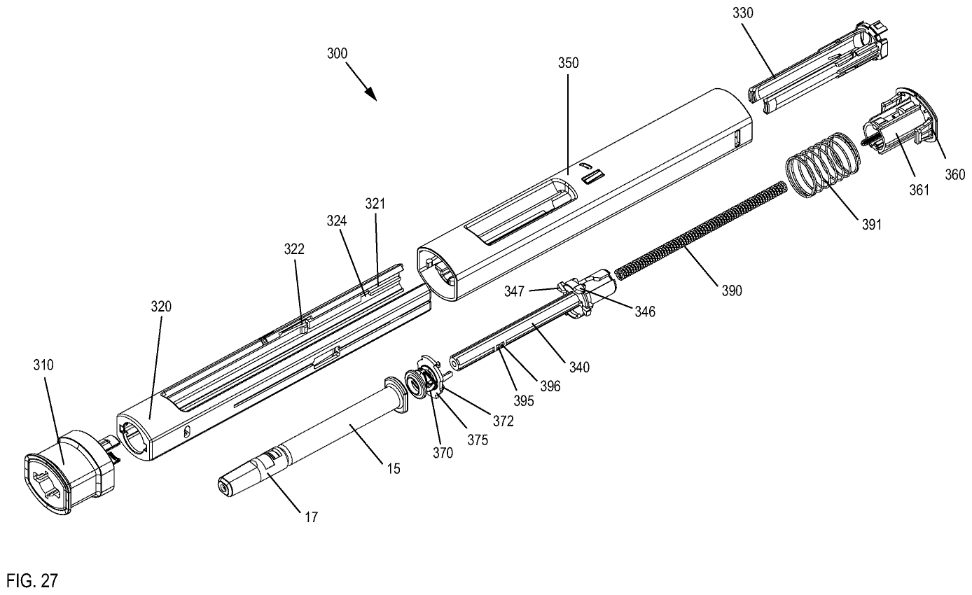

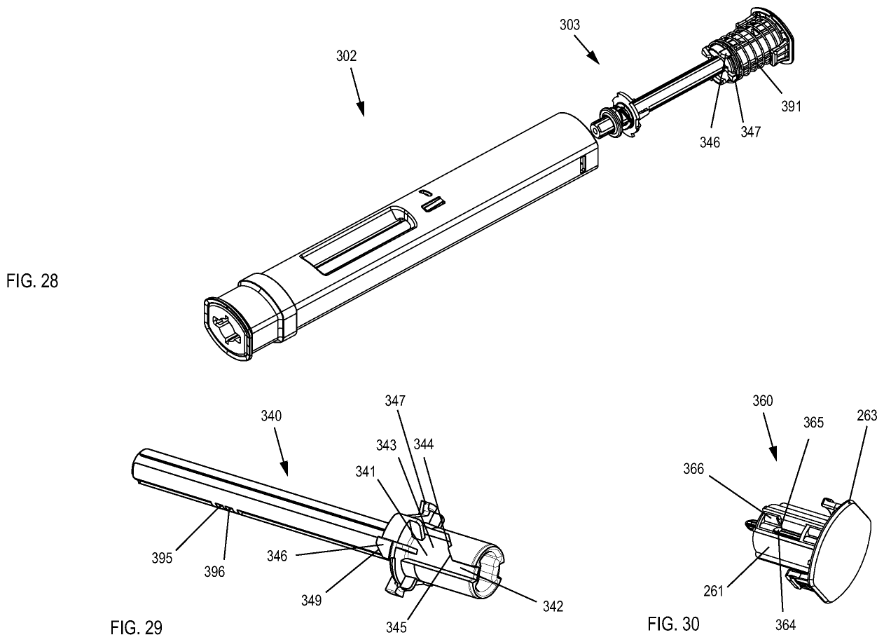

FIG. 27 depicts a perspective exploded view of an injection device according to a fourth embodiment of the present disclosure in form of an autoinjector 300. The autoinjector 300 includes a pre-assembled syringe unit 302 and a pre-assembled drive unit 303 as shown in FIG. 28.

The pre-assembled syringe unit 302 includes a sleeve-shaped housing 350, a needle cover sleeve 320, a cap 310, a syringe holder 330 and the prefilled syringe (PFS) 15, see FIG. 27. A rigid needle shield (RNS) 17 is initially mounted on the injection needle 18. The housing, the needle cover sleeve, the cap and the syringe holder are injection-molded plastic parts that may be made of polypropylene (PP).

The pre-assembled drive unit 303 includes a supporting member 360, a hollow plunger rod 340, an injection spring 390 to move the plunger rod 340 in the dispensing direction, a cover sleeve spring 391 adapted to bias the needle cover sleeve 320, and a lock member 370 coupled to the plunger rod 340. The supporting member, the plunger rod and the lock member are injection-molded plastic parts and may be made of polyoxymethylene (POM).

For the assembly of the pre-assembled syringe unit 302 the needle cover sleeve 320 is inserted into the housing 350. As the housing 350 has flattened sides and has thus has a non-circular cross-section, the needle cover sleeve 320 cannot rotate relative to the housing but is movable along the longitudinal axis. The cap 310 is mounted on a distal end of the needle cover sleeve 320.

As a next assembly step, the syringe holder 330 is inserted from a proximal side into the housing 350 but has not yet moved into a distal end position. After the above assembly step, the syringe unit 302 is in its pre-assembled state and all components of the syringe unit are fixed and cannot be detached or separated unless a snap-fit connection is released.

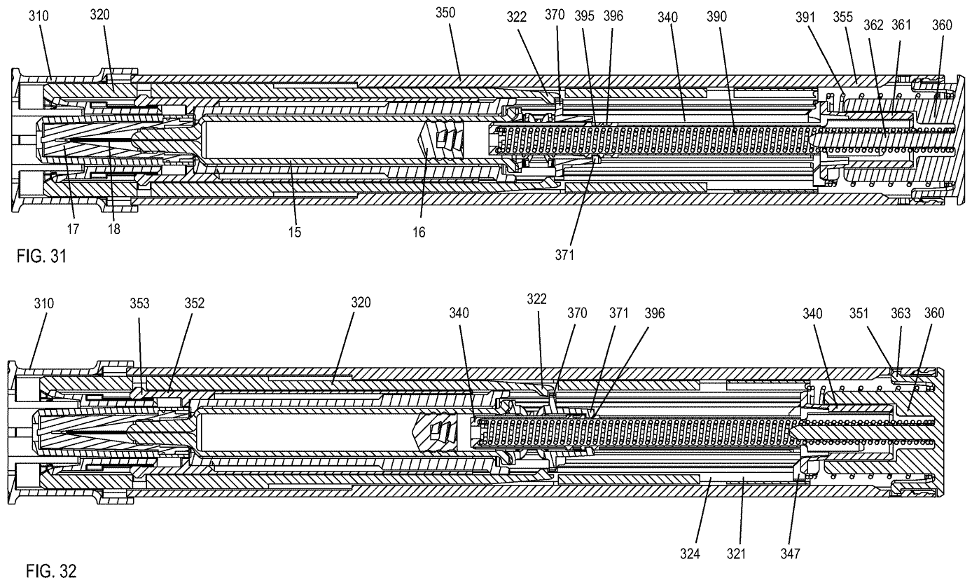

At the drug manufacturer site or at the assembly site, the prefilled syringe 15 with its RNS 17 is inserted from the proximal side into the syringe holder 330. The syringe holder 330 together with the syringe 12 is then moved into its final distal position within the housing 350 as shown in FIG. 31.

For the pre-assembly of the drive unit 303 the injection spring 390 in form of a spiral spring is axially inserted into a bore or opening of the sleeve-shaped portion 361 of the supporting member 360 such that the windings of the spring 390 are located radially between an inner guiding pin 362 and the inner wall of the sleeve-shaped portion 361 as shown in FIG. 31. The cover sleeve spring 391 is mounted onto the sleeve-shaped portion 361 and then the plunger rod 340 is axially inserted into a distal opening of the sleeve-shaped portion.

FIG. 29 shows a perspective view of the plunger rod 340 alone, and FIG. 30 depicts a perspective view of the supporting member 360 alone. The plunger rod 340 includes in a proximal portion two oppositely arranged guiding tracks 341. Each guiding track 341 includes an axial groove section 342 and a tangentially extended section 343. Section 343 includes a distally oriented wall including a sloped lock surface 344 and an adjacent sloped release surface 345.

As shown in FIG. 30 the supporting member 360 includes on its sleeve-shaped portion 361 two oppositely arranged and radially inwards protruding cams 366 each having a sloped lock surface 364 on one side and a sloped release surface 365 on the other side. The two surfaces 364, 365 of each cam 366 are proximally oriented and arranged adjacent to each other such that each cam 366 is arrowhead shaped.

During assembly the cams 366 are inserted into the axial groove sections 342 and then the plunger rod 340 is rotated relative to the supporting member 360 such that the cams 366 enter the sections 343. As the plunger rod 340 is biased by the injection spring 390 in the distal direction, the sloped lock surface 344 of the plunger rod and the sloped lock surface 364 of the supporting member are pressed against each other. As the lock sloped surface 344 of the plunger rod is oriented away from a guide track inlet, the plunger rod 340 is reliably held in place and coupled to the supporting member. That means during the assembly the plunger rod 340 is connected to the supporting member 360 by an axial and a subsequent rotational movement and thus the plunger rod 340 is connected to the supporting member 360 by a bayonet connection.

As a final step, the lock member 370 is mounted onto the plunger rod 340 from a distal side and shifted proximally until two oppositely arranged clamping arms 371 of the lock member 370 snap into a first recess 395 in the plunger rod, see FIG. 31. The plunger rod includes a first distal recess 395 and a second proximal recess 396 which are also shown in FIG. 29. The lock member 370 is thus fixed relative to the plunger rod 340 and cannot rotate or axially move relative to the plunger rod.

The drive unit 303 is then in its pre-assembled state as shown in FIG. 28. All components of the pre-assembled drive unit 303 are fixed and cannot be detached or separated unless a snap-fit connection is released. That is, the cover sleeve spring 391 is compressed and held in place by two oppositely arranged first guiding cams 346 and two oppositely arranged second guiding cams 347 of the plunger rod 340. The latter is held on the supporting member by the above-described bayonet connection.

The pre-assembled syringe unit 302 with the inserted syringe 15 is then connected to the pre-assembled drive unit 303. The plunger rod 340 with the attached lock member 370 and the sleeve-shaped portion 361 of the supporting member is inserted into a connecting portion 355 (FIG. 31) of the housing 350 from a proximal side.

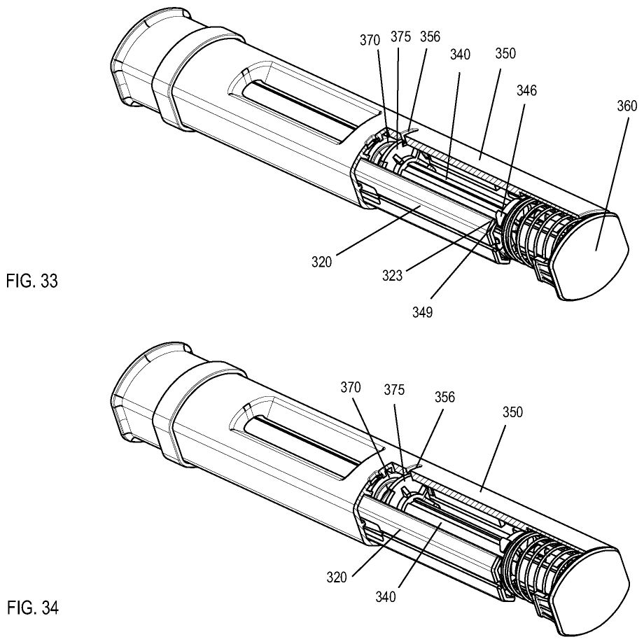

FIG. 31 and FIG. 33 depict a state in which the drive unit 303 is partly inserted but not yet completely connected to the syringe unit 302. FIG. 31 shows a sectional view of the autoinjector and FIG. 33 depicts a corresponding perspective view. In FIG. 33 one half of the proximal housing section is not drawn to allow an inspection of the inner parts of the autoinjector.

As best shown in FIG. 31, the lock member 370 is already in a distal end position as it encounters a proximal end of locking arms 322 of the needle cover sleeve 320. However, the plunger rod 340 is not yet completely inserted and, as shown in FIG. 33, sloped walls 349 of the first guiding cams 346 abut proximal edges 323 of the needle cover sleeve 320. The lock member clamping arms 371 engage the first recesses 395 in the plunger rod as shown in FIG. 31.

In FIG. 33, a flange 375 of the lock member 370 is axially aligned with a locking recess 356 in the housing 350. In the position shown in FIG. 33 the flange 375 has not yet rotated into the locking recess 356.

When the drive unit 303 is further inserted into the syringe unit housing 350, the sloped walls 349 are further pressed against the edges 323 and subsequently the edges slide along the sloped walls 349. This causes the plunger rod 340 to move slightly proximally and rotate relative to the supporting member 360 and to the housing 350, while the proximal end of the needle cover sleeve 320 can move proximally of the cams 346 and is now in direct contact with the cover sleeve spring 391

Concurrently, the distal movement of the plunger rod during insertion moves the plunger rod 340 relative to the lock member 370 as the latter abuts the cover sleeve locking arms 322. Due to the relative movement, the snap-fit connection between the clamping arms 371 and the first recess 395 (see FIG. 31) is released and the clamping arms 371 snap in the second recesses 396 as shown in FIG. 32. The lock member 370 is thus rotationally and axially locked to the plunger rod 340.

FIG. 32 and the corresponding perspective view in FIG. 34 show a final assembly state when the drive unit 303 is completely inserted into the housing 350. As can be seen in FIG. 34, the lock member 370 has been rotated together with the plunger rod 340 relative to the housing 350 into a final position. As depicted in FIG. 32 two oppositely arranged snap elements 363 of the supporting member have snapped into corresponding recesses 351 in the housing and thus the supporting member 360 is fixed to the housing 350 and cannot move relative thereto.

The above-mentioned movement of the plunger rod 340 relative to the supporting member 360 during assembly causes the cams 366 of the supporting member 360 to move out of the section 343 of the plunger rod 340 (FIGS. 29 and 30). Therefore, the bayonet connection is partly released and the sloped release surface 365 of the cam engages the sloped release surface 345 of the plunger rod. As the plunger rod 340 is biased by the injection spring 390, the two sloped surfaces 365, 345 are pressed against each other but the plunger rod cannot rotate because it is held in place by a set of second guiding cams 347, which abut linear guide rails 321 on the needle cover sleeve 320, see FIG. 32. The plunger rod 340 includes two oppositely arranged second guiding cams 347, which are also shown in FIGS. 28 and 29. The second guiding cams 347 are positioned at the same axial position but are rotated 90° around the longitudinal axis relative to the first guiding cams 346. Correspondingly, the needle cover sleeve 320 includes two oppositely arranged linear guide rails 321 also shown in FIG. 27. The biased plunger rod 340 is thus held in place and cannot move. Instead of having the guide rails on the needle cover sleeve and the corresponding cams on the plunger rod, guide rails may be arranged on the plunger rod and guide cams on the needle cover sleeve. The axial extension of the rails is understood to be superior to those of the cams, and the relative arrangement prior to triggering the injection defining an axial distance between the distal end of the rail or cam on the needle cover sleeve and the proximal end of the cam or rail on the plunger rod inferior to the retraction distance.

Furthermore, as shown in FIG. 34 due to the rotation of the plunger rod 340, the flange 375 of the lock member 370 is inserted into the locking recess 356 in the housing 350. That means the plunger rod 340 is thus axially locked to the housing by the lock member 370 such that the plunger rod cannot move along the longitudinal axis in this rotational orientation.

The assembled autoinjector 300 is then in a ready to use state. The cap 310 is mounted on the distal end and the securing arms 352 of the housing as shown in FIG. 32 prevent a proximal retraction movement of the needle cover sleeve 320 as described with respect to the first embodiment.

Before an injection, the user removes the cap 310. The RNS 17 is removed together with the cap 310, as hooks of the cap engage a proximal rim of the RNS. When the cap 310 is removed from the needle cover sleeve 320 the securing arms 352 of the housing 350 are no longer prevented from being deflected radially inwards. That means if the user pushes the autoinjector against the injection site, the needle cover sleeve 320 can move proximally thereby deflecting knob ends 353 (FIG. 32) of the securing arms 352. That means the securing arms 352 can deflect radially inwards to release the needle cover sleeve 320.

Therefore, the cover sleeve 320 moves from its covering position into its retracted position. This is enabled as cutouts 372 (see FIG. 27) in the lock member 370 are rotationally aligned with locking arms 322 of the needle cover sleeve such that the locking arms can pass the lock member 370.

During the proximal retraction movement, the second guiding cams 347 disengage the linear guide rail 321 on the needle cover sleeve and enter into an opening or cavity 324 (FIGS. 32 and 27). That means the plunger rod 340 is no longer rotationally guided. This in turn means that the sloped release surfaces 345 of the plunger rod 340 and the release surfaces 365 of the support member can slide along each other, causing the plunger rod 340 to rotate relative to the supporting member 360. This also causes the snap arms 371 of the supporting member 370 to rotate out of the second recesses 396 of the plunger rod. Thereby, the cam 366 gets into the axial groove section 342 in the guide track 341 and therefore the bayonet connection is released. The plunger rod 340 can thus be moved in the dispensing direction by force of the biased injection spring 390.

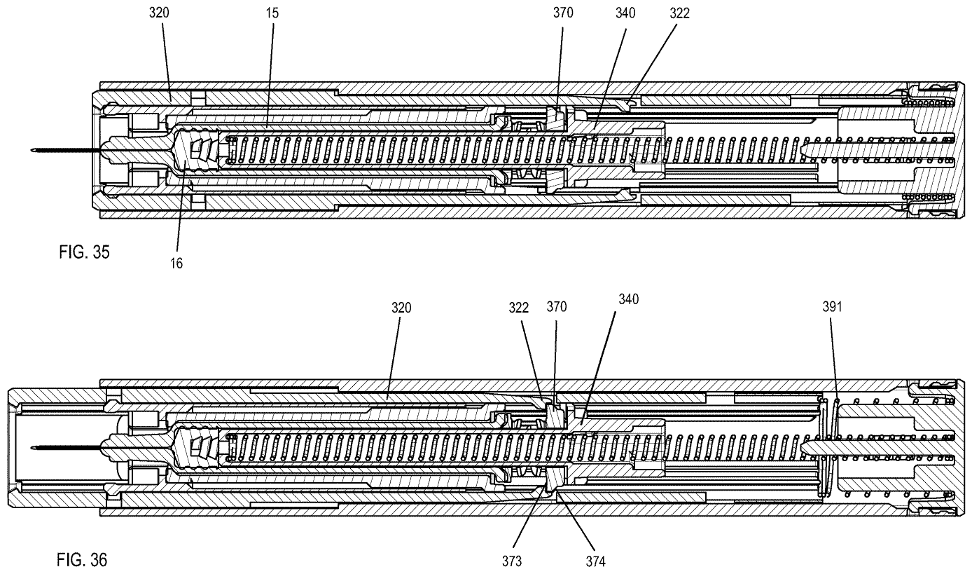

In the state shown in FIG. 35, the needle cover sleeve 320 is in its retracted position and the autoinjector 300 is in a state where the plunger rod 340 is in a distal end position and thus the piston 16 is in a distal end position and the drug is completely dispensed.

As the plunger rod 340 is rotated prior to the distal injection movement, the lock member 370 is rotated with it, relative to the housing 350 and relative to the needle cover sleeve 320. That means after the needle cover sleeve is moved proximally and after rotation of the plunger rod the cutouts 372 in the flange 375 as shown in FIG. 27 for the locking arms 322 are no longer rotationally aligned with the locking arms.

Once the injection is completed the user removes the autoinjector 300 from the injection site. Due to the cover sleeve spring 391 the needle cover sleeve 320 is moved from the retracted position back into its distal covering position. This state of the autoinjector 300 is shown in FIG. 36.

As the plunger rod 340 has been rotated during needle cover retraction movement as previously described, the flange 375 of the lock member 370 is rotationally aligned with the locking arms 322. As shown in FIG. 36 the flange 375 has a stop surface 373 on a distal side and a ramp 374 on the proximal side. Before the needle cover sleeve 320 reaches its final covering position again, the locking arms 322 are radially deflected outwards by the ramps 374 of the lock member 370 such that the locking arms 322 can pass and move in the distal direction. As the flange 375 includes the stop surface 373 on the distal side, the arms 322 encounter the stop surface 373 and thus the needle cover sleeve 320 is prevented from being moved again towards the retracted position. The needle cover sleeve 320 is thus locked in its covering position (needle cover lock).

The autoinjector, according to the fourth embodiment, may be disassembled and the parts made of the same material can be discarded together. Alternatively, and as described with respect to the first embodiment, the autoinjector may be reused after a reset procedure.

While the disclosure has been described in detail in the drawings and foregoing description, such description is to be considered illustrative or exemplary and not restrictive. Variations to the disclosed embodiments can be understood and effected by those skilled in the art and practising the claimed invention, from a study of the drawings, the disclosure, and the appended claims. In the claims, the word “comprising” does not exclude other elements or steps, and the indefinite article “a” or “an” does not exclude a plurality. The mere fact that certain elements or steps are recited in distinct claims does not indicate that a combination of these elements or steps cannot be used to advantage, specifically, in addition to the actual claim dependency, any further meaningful claim combination shall be considered disclosed.

### LIST OF DESIGNATIONS

### First Embodiment: FIGS. 1 to 8

| | |
|---|---|
| 1 | autoinjector |
| 2 | syringe unit |
| 3 | drive unit |
| 10 | cap |
| 11 | remover sleeve |
| 12 | hooks |
| 15 | prefilled syringe |
| 16 | piston |
| 17 | rigid needle shield |
| | RNS |
| 18 | injection needle |
| 20 | needle cover sleeve |
| 21 | ledge |
| 22 | distal snap element |
| 23 | securing rip |
| 24 | sloped surfaces |
| 25 | proximal snap element |
| 26 | inner surface |
| 30 | syringe holder |
| 31 | rim |
| 40 | plunger rod |
| 41 | distal recess |
| 42 | proximal recess |
| 43 | sloped surface |
| 44 | release surface |
| — | |
| 50 | housing |
| 51 | snap element |
| 52 | securing arms |
| 53 | knob end |
| 54 | sloped surface |
| 55 | connecting portion |
| 60 | supporting member |
| 61 | sleeve-shaped |
| | portion |
| 62 | guiding pin |
| 63 | end wall |
| 64 | distal recess |
| 65 | distal retaining arms |
| 66 | distal knob |
| 67 | proximal retaining |
| | arms |
| 68 | proximal knob |
| 69 | sloped surface |
| 70 | lock sleeve |
| 71 | ledge |
| 72 | sloped surface |
| 75 | locking arms |
| 76 | cam end |
| 77 | recess |
| 78 | holding arms |
| 79 | holding cams |
| 80 | needle cover sleeve |
| 81 | recess |
| 82 | supporting member |
| 84 | lock sleeve |
| 85 | locking arms |
| 86 | cam end |
| 87 | protrusion |
| 88 | first recess |
| 89 | second recess |
| 90 | injection spring |
| 91 | cover sleeve spring |
| 92 | release surface |
| 93 | ramp |
| 94 | recess |
| 95 | rounded corner |
| 96 | support surface |
| 97 | proximal recess |

### Second Embodiment: FIGS. 9 to 16

| | | | |
|---|---|---|---|
| 100 | autoinjector | 150 | housing |
| 102 | syringe unit | 151 | snap element |
| 103 | drive unit | 152 | recess |
| 110 | cap | 153 | sloped contact surface |
| 15 | prefilled syringe | 155 | connecting portion |
| 16 | piston | 160 | supporting member |
| 17 | rigid needle shield RNS | 161 | sleeve-shaped portion |
| 18 | injection needle | 162 | guiding pin |
| 120 | needle cover sleeve | 163 | end wall |
| 126 | U-shaped channel | 164 | L-shaped snap element |
| 127 | angled wall | 165 | radial cam |
| 128 | stop wall | 166 | radial snap element |
| 130 | syringe holder | 167 | retaining arm |
| 131 | flange | 168 | protrusion |
| 132 | nose | 169 | guiding cam |
| 133 | nose | 170 | spring element |
| 140 | plunger rod | 171 | opening |
| 141 | recess | 190 | injection spring |
| 142 | recess snap element | 191 | cover sleeve spring |
| 143 | holding ribs | 192 | nose |
| 144 | end surface | 193 | sloped surface |
| 145 | arrowhead | 196 | support surface |
| 146 | connecting portion | | |

### Third Embodiment: FIGS. 17 to 26 and 37 to 39

| | | | | | |
|---|---|---|---|---|---|
| 200 | autoinjector | 260 | supporting member | 240′ | plunger rod |
| 202 | syringe unit | 261 | sleeve-shaped | 241′ | recess |
| 203 | drive unit | | portion | 246′ | |
| 210 | cap | 262 | guiding pin | 246″ | |
| 211 | tongues | 263 | end wall | 260′ | supporting member main body |
| 212 | hooks | 264 | snap arm | 260″ | supporting member insert |
| 15 | prefilled syringe | 265 | retaining arms | 263′ | insert end wall |
| 16 | piston | 266 | protrusion | 264′ | insert snap arms |
| 17 | rigid needle shield | 267 | cam | 265′ | snap arms |
| | RNS | 268 | angled wall | 266′ | guiding pin |
| 18 | injection needle | 269 | axial rib | 267′ | stop walls |
| 220 | needle cover sleeve | 270 | lock sleeve | 270′ | lock sleeve |
| 221 | extension | 271 | retaining nose | 274′ | locking surface |
| 230 | syringe holder | 272 | sloped surface | 276′ | first axial groove |
| 231 | cam | 273 | stop surface | 276″ | securing cam |
| 240 | plunger rod | 274 | locking surface | 277′ | second axial groove |
| 241 | recess | 276 | first axial groove | 278′ | angled section |
| 242 | sloped surface | 277 | second axial groove | 279′ | sawtooth |
| 245 | plunger rod | 278 | angled section | 290′ | injection spring |
| 246 | saw teeth | 279 | sawtooth | 291′ | cover sleeve spring |
| 247 | plunger rod | 280 | spring element | 292′ | deflectable arm |
| 248 | guiding pin | 281 | opening | 293′ | nose |
| 250 | housing | 285 | locking arms | | |
| 251 | recess | 290 | injection spring | | |
| 252 | recess | 291 | cover sleeve spring | | |
| 255 | connecting portion | 292 | deflectable arm | | |
| | | 293 | nose | | |
| | | 294 | supporting member | | |
| | | 295 | supporting member | | |
| | | 296 | deflectable arm | | |
| | | 297 | nose | | |

### Fourth Embodiment: FIGS. 27 to 36

| | | | |
|---|---|---|---|
| 300 | autoinjector | 350 | housing |
| 302 | syringe unit | 351 | recess |
| 303 | drive unit | 352 | securing arms |
| 15 | prefilled syringe | 353 | knob end |
| 16 | piston | 355 | connecting portion |
| 17 | rigid needle shield RNS | 356 | locking recess |
| 18 | injection needle | 360 | supporting member |
| 310 | cap | 361 | sleeve-shaped portion |
| 320 | needle cover sleeve | 362 | guiding pin |
| 321 | linear guide rail | 363 | snap element |
| 322 | locking arms | 364 | lock surface |
| 323 | edge | 365 | release surface |
| 324 | cavity | 366 | cam |
| 330 | syringe holder | 370 | lock member |
| 331 | rim | 371 | clamping arms |
| 340 | plunger rod | 372 | cutout |
| 341 | guide track | 373 | stop surface |
| 342 | axial groove section | 374 | ramp |
| 343 | tangentially extended | 375 | flange |
| | section | 390 | injection spring |
| 344 | lock surface | 391 | cover sleeve spring |
| 345 | release surface | 395 | first recess |
| 346 | first guiding cam | 396 | second recess |
| 347 | second guiding cam | 397 | support surface |
| 349 | sloped wall | | |

What is claimed is:

1. A method of assembling an injection device for dispensing a liquid drug from a syringe through an injection needle from a pre-assembled drive unit and a pre-assembled syringe unit, wherein the syringe unit comprises:
a housing defining a longitudinal axis; and
a needle cover movable along the longitudinal axis between a covering position
and a retracted position in which the needle protrudes from the needle cover, and wherein the drive unit comprises:
a plunger rod;
a spring member adapted to bias the plunger rod in a dispensing direction;
a supporting member adapted to support or guide the spring member or the plunger rod; and
a first and a second retaining element, wherein each retaining element is configured to be arranged in a holding position in which the first or second retaining element holds the plunger rod biased in place relative to the supporting member, and in a release position in which the first or second retaining element does not hold the biased plunger rod in place,
the method comprising the steps of:
inserting the drive unit into the syringe unit, wherein the first retaining element is in the holding position and the biased plunger rod is exclusively held in place by the first retaining element; and
moving the housing and the needle cover relative to the supporting member to operatively couple the needle cover to the second retaining element and to move the first retaining element out of the holding position, such that, when the injection device is completely assembled, the second retaining element is in the holding position and the biased plunger rod is exclusively held in place by the second retaining element when the needle cover is in the covering position.

2. The method of claim 1, further comprising a final step of establishing a snap fit connection between the supporting member and the housing.

3. The method of claim 1, further comprising, prior to inserting the drive unit into the syringe unit, the steps of:
separating the syringe unit and the drive unit previously and provisionally connected for transporting purposes; and
inserting the syringe into the syringe unit.

4. An injection device for dispensing a liquid drug from a syringe through an injection needle, the injection device comprising a pre-assembled drive unit assembled with a pre-assembled syringe unit, wherein the pre-assembled syringe unit comprises:
a housing defining a longitudinal axis; and
a needle cover movable along the longitudinal axis relative to the housing between a needle covering position and a retracted position in which the needle protrudes from the needle cover, and
wherein the pre-assembled drive unit comprises:
a plunger rod;
a spring member adapted bias the plunger rod in a dispensing direction;
a supporting member adapted to support or guide the spring member or the plunger rod; and
a first and a second retaining element, wherein each retaining element is configured to be arranged in a holding position in which the first or second retaining element holds the plunger rod biased in place relative to the supporting member and in a release position in which the first or second retaining element does not hold the biased plunger rod in place,
wherein prior to assembly of the injection device the pre-assembled drive unit and the pre-assembled syringe unit are separated from each other, and the first retaining element is in the holding position and the biased plunger rod is held in place by the first retaining element,
wherein during connection of the syringe unit to the drive unit a movement of the housing and of the needle cover relative to the supporting member causes the first retaining element to move out of the holding position,
wherein, when the injection device is completely assembled, the drive unit is connected to the syringe unit and the needle cover is operatively coupled to the second retaining element, the first retaining element is in the release position, and the second retaining element is in the holding position such that the biased plunger rod is exclusively held in place by the second retaining element when the needle cover is in the covering position.

5. The injection device according to claim 4, wherein the supporting member further comprises the first and the second retaining element, and wherein the supporting member comprises at least one of:
a connecting element adapted to connect the pre-assembled drive unit to the pre-assembled syringe unit;
a guiding element adapted to guide the plunger rod along a longitudinal direction; and
a support surface for supporting the spring member at a proximal end of the spring member.

6. The injection device according to claim 5, wherein the supporting member further comprises a sleeve-shaped portion comprising a circumferential wall defining a bore, wherein the biased plunger rod is at least partly arranged inside the bore.

7. The injection device according to claim 6, wherein the first and second retaining elements are arranged on the wall.

8. The injection device according to claim 4, wherein the first retaining element is a deflectable arm extending in a longitudinal direction with a retaining portion on a free end.

9. The injection device according to claim 4, wherein the plunger rod is hollow defining a cavity and wherein the spring member is arranged inside the cavity, and wherein the supporting member comprises a guiding pin extending along the longitudinal axis inside the spring member and inside the cavity of the plunger rod to guide the spring member.

10. The injection device according to claim 4, wherein the pre-assembled syringe unit further comprises a syringe holder, and wherein the needle cover is coaxially arranged inside the housing and the syringe holder is arranged inside the needle cover.

11. The injection device according to claim 4, wherein the housing comprises a release surface adapted to move the first retaining element out of the holding position and into the release position during connection of the pre-assembled syringe unit with the pre-assembled drive unit.

12. The injection device according to claim 10, wherein in an assembled state, proximally to the syringe holder the housing comprises a connecting portion, which extends along the longitudinal axis at least a half length of an axial length of the syringe holder, and wherein the connecting portion is adapted to accommodate and be connected to the drive unit.

13. The injection device according to claim 10, wherein the drive unit further comprises a lock sleeve movable relative to the supporting member adapted to lock, in the assembled state, the second retaining element in the holding position such that the second retaining element cannot be brought into the release position.

14. The injection device according to claim 10, wherein the supporting member comprises an elastic element on a distal end adapted to bias the syringe inside the syringe holder in an assembled state of the injection device, wherein the elastic element is integrally formed in the supporting member as a monolithic part.

15. The injection device according to claim 4, wherein the supporting member, the first and second retaining element and the plunger rod are constructed of a first material, and wherein the housing and the needle cover are made of a second material, different from the first material.

16. An injection device for dispensing a liquid drug from a syringe through an injection needle, the injection device comprising a pre-assembled drive unit assembled with a pre-assembled syringe unit, wherein the pre-assembled syringe unit comprises:
a housing defining a longitudinal axis; and
a needle cover movable along the longitudinal axis relative to the housing,
wherein the pre-assembled drive unit comprises:
a plunger rod;
a spring member adapted to bias the plunger rod in a dispensing direction;
a supporting member; and
a first and a second retaining element, wherein each retaining element is configured to be arranged in a holding position in which the first or second retaining element holds the plunger rod biased in place relative to the supporting member and in a release position in which the first or second retaining element does not hold the biased plunger rod in place,
wherein prior to assembly of the injection device, the pre-assembled drive unit and the pre-assembled syringe unit are separated from each other, and the first retaining element is in the holding position and the biased plunger rod is exclusively held in place by the first retaining element,
wherein, when the injection device is completely assembled, the drive unit is connected to the syringe unit and the needle cover is operatively coupled to the second retaining element, the first retaining element is in the release position, and the second retaining element is in the holding position such that the biased plunger rod is exclusively held in place by the second retaining element when the needle cover is in a covering position.

17. The injection device according to claim 16, wherein the supporting member further comprises the first and the second retaining elements, and wherein the supporting member comprises at least one of:
a connecting element adapted to connect the pre-assembled drive unit to the pre-assembled syringe unit;
a guiding element adapted to guide the plunger rod along a longitudinal direction; and
a support surface for supporting the spring member at a proximal end of the spring member.

18. The injection device according to claim 16, wherein the plunger rod is hollow defining a cavity, wherein the spring member is arranged inside the cavity, and wherein the supporting member comprises a guiding pin extending along the longitudinal axis inside the spring member and inside the cavity of the plunger rod to guide the spring member.

19. The injection device according to claim 16, wherein the pre-assembled syringe unit further comprises a syringe holder, and wherein the needle cover is coaxially arranged inside the housing and the syringe holder is arranged inside the needle cover.

20. The injection device according to claim 16, wherein the housing comprises a release surface adapted to move the first retaining element out of the holding position and into the release position during connection of the pre-assembled syringe unit with the pre-assembled drive unit.
